(19) United States
(12) Patent Application Publication
(10) Pub. No.: US 2009/0221497 A1
(43) Pub. Date: Sep. 3, 2009

(54) Immunodynamic complexes and methods for using and preparing such complexes

(76) Inventors: Jesse A. Stoff, Tucson, AZ (US); David L. Bergsma, Scottsdale, AZ (US); Harry Adon Willett, Phoenix, AZ (US); Paul James Weighner, Lansing, IA (US); Mark Joseph Weighner, Eaton, CO (US)

Correspondence Address: COVINGTON & BURLING, LLP;ATTN: PATENT DOCKETING, 1201 PENNSYLVANIA AVENUE, N.W., WASHINGTON, DC, 20004-2401, US

(21) Appl. No.: 12/289,092

(22) Filed: Oct. 20, 2008

Related U.S. Application Data
(63) Continuation of application No. 11/122,231, filed on May 5, 2005, now abandoned.
Application No. 11/122,231 is a continuation-in-part of application No. 10/838,539, filed on May 5, 2004, now abandoned.

(51) Int. Cl. A61K 38/17

(52) U.S. Cl. 514/12

(57) ABSTRACT

The present invention is directed to immunodynamic complexes that, in embodiments of the invention, are surprisingly antimicrobial and immunoactive. By “immunoactive,” it is meant that such compositions are capable of modulating, stimulating and repairing the immune system. Moreover, in embodiments of the invention, an immunodynamic complex is capable of supporting, maintaining and/or enhancing the structure and function of the immune system. In addition, this invention is directed to a method for preparing and using such compositions. Embodiments of the immunodynamic complexes of this invention are prepared from lacteal secretions derived from ungulates, such as cows.

### FIELD OF THE INVENTION

This invention pertains to immunodynamic complexes (i.e., antimicrobial and immunoactive compositions) and methods for using and preparing such complexes. In particular, such complexes can be derived from lacteal secretions.

### BACKGROUND OF THE INVENTION

A filtrate of colostrum and whey from antigen infused dairy cows has been used since the late 1950s by humans and animals. Empirical and anecdotal evidence since the introduction of this material indicates that it has properties significantly different from typical colostrum and whey products. From the beginning, this material appeared to restore immune function in people and animals that were suffering from conditions where the immune system was not capable of providing protection and/or correction from the development of acute, chronic and degenerative diseases. Early researchers of antigen infusion developed standardized methodologies to produce consistent material that contained molecules that were unidentified but thought to exist in the filtrate.

U.S. Pat. No. 3,376,198 to Petersen et al. describes a method for providing a milk-type product derived from ungulates (particularly cows, goats, sheep, etc.) that has been fortified with naturally occurring antibodies in what are said to be therapeutically significant concentrations. The high concentrations of a specific antibody in such milk-type products are produced against an antigen by introducing that antigen into the udder of the animal during pregnancy. The patent explains that this may be done by infusing the antigen through the teat canals (i.e., “infusion”), or by injecting the antigen hypodermically through the wall of the udder close to the base of each teat, or wherever the injected antigen is sure to reach the cistern of the udder (i.e., “injection”). The antigen may be introduced at weekly intervals while the animal is pregnant. Declining antibody concentrations may be increased by periodically introducing booster shots of the selected antigen into the udder during the lactating period. The booster shots may be given intravenously, intramuscularly, subcutaneously or may be made into the duct system of the udder through the teat meatus and into the grand cistern.

U.S. Pat. No. 4,402,938 to Collins et al. describes a food product that can be used as a nutritional supplement for animals and is made of whey obtained from either colostrum or milk as it comes from selected cows or other ungulates, and contains an active fraction having a molecular weight on the order of 1200 or less. Both here and throughout this application, by “cow” we mean a cow from the bovine species. This active fraction is produced by taking an antigen-like material and introducing it into the udder of an ungulate in an aseptic manner, two or three times, at weekly intervals during the last month of gestation. This can be accomplished by using a sterile syringe and hypodermic needle and injecting the material into the side of the udder. Alternatively, the material can be introduced through the teat canal using a sterile syringe and the blunt plastic needle inserted through the orifice of the teat into the cistern. Although this patent was unable to identify the actual identity of the disclosed food product, its value was said to be proven by extensive and conclusive tests. The patent further indicates that the product could be processed by aseptically bottling for direct consumption or by freeze-drying to produce a powder form.

Further to these patents, a traditional antigen infusion technique has developed and has been in place for some time, as noted earlier. However, the products of such antigen infusion customarily are blended from several different cows where each animal was given a different antigen infusion. These blends have been used essentially only to support and normalize immune function, that is, for much the same reasons as one might take vitamins or dietary supplements.

For example, one product of blended lacteal secretions influenced by antigen infusion that is currently on the market is sold under the trade name Ai/E¹⁰® by Quantum Research, Inc., headquartered in Scottsdale, Ariz. (“Ai/E¹⁰®”). Ai/E¹⁰® is a blend of refined lacteal secretions influenced by antigen infusion that contains an array of immuno-active proteins and other immunological factors that are less than or equal to 100 kD in size and that supports the structure and function of the immune system. This product can be referred to as a refined lacteal complex (“RLC”), by which is meant a lacteal secretion (i.e., colostrum or milk), from an ungulate, that has been refined.

However, the products made from traditional antigen infusion techniques have not always been considered effective, and have not been used as therapeutic compositions, for example, as antimicrobial agents.

It would be highly desirable to develop new antimicrobial and immunoactive compositions that can be used to treat living organisms, including humans and animals. This is particularly the case given the increasingly serious problem of antibiotic resistance and the lack of new antimicrobial substances to treat new infectious diseases, whether they be caused by viruses, bacteria, mycobacteria, protozoa, spirochetes or other microbes. It also would be very helpful to develop a technique by which new antimicrobial and immunoactive compositions could be developed rapidly to combat new or even known infectious diseases and restore immune function in immune-compromised patients. An immunoactive composition that helps restore immune function and that would be capable of conferring some degree of immunity to a patient could also be beneficial. Moreover, an immunoactive composition that could maintain, support and enhance the structure and function of the immune system could also be beneficial.

### SUMMARY OF THE INVENTION

An object of our invention is the development of new antimicrobial and immunoactive compositions, and a novel method of making such compositions from a lacteal secretion.

Yet another object of our invention is the development of an immunoactive composition that could maintain, support and enhance the structure and function of the immune system.

Still another object of our invention is to provide an assay for quantification of processing procedures used to make such antimicrobial and immunoactive compositions and the qualification of such resulting products.

Consequently, in one aspect, our invention provides a composition capable of providing an antimicrobial benefit or reestablishing cytokine pathways when administered to a living organism in a pharmaceutically effective dose, wherein the composition comprises: (a) a pharmaceutically effective combination comprising: (i) an amount of granulysins; (ii) an amount of transfer factors; (iii) an amount of defensins; and (iv) an amount of mini-cytokines; and (b) a pharmaceutically acceptable carrier. The granulysins, transfer factors and defensins in the combination are specific to a selected pathogen.

In another aspect, our invention provides an immunodynamic complex derived from a lacteal secretion of an ungulate, wherein the lacteal secretion comprises at least about 100 times greater amounts of molecular material in the less than about 100 kD range than is found in a normal lacteal secretion (i.e., normal milk). The molecular material contains, among other components, granulysins, transfer factors and defensins.

In yet another aspect, our invention provides a method of preparing an immunodynamic complex derived from a lacteal secretion of an ungulate involving a number of steps, including: preparing a mixture of an amount of an antigen together with an amount of a cytokine mix; gun-inoculating the antigen/cytokine mixture into at least one quarter of an ungulate's udder; harvesting a lacteal secretion from the ungulate's udder starting about 72 hours after inoculating; and obtaining an extract of molecules from the lacteal secretion having a molecular weight of less than or equal to about 100 kD.

In still another aspect, our invention provides a mixture for infusion into a cow containing: between about 0.1 cc and about 20 cc of a 10% solution of fetal calf serum and phosphate buffered saline, or a 1% solution of bovine albumin and phosphate buffered saline; between about 0.1 cc and about 20 cc of an antigen; between about 0.001 cc and about 2 cc Alpha-TNF; between about 0.001 cc and about 2 cc G-INF; between about 0.001 cc and about 2 cc IL-8; and between about 0.001 cc and about 2 cc GM-CSF.

In an additional aspect, our invention provides a lacteal secretion obtained from an ungulate that, upon analysis, reveals a molecular shift. This molecular shift reflects that the lacteal secretion contains at least about 100 times greater amounts of molecular material in the less than about 100 kD range than is found in a normal lacteal secretion (i.e., normal milk), and in that the lacteal secretion includes granulysins, transfer factors and defensins.

In yet another aspect, our invention provides a method for testing the quality of an antigen, for example in preparing an immunodynamic complex, as measured by whether the antigen is immunologically recognizable, comprising: preparing two cell culture samples each comprising white blood cells; adding the antigen to one cell culture sample; labeling the cells in each cell culture sample with CD 25, CD 69 or any other activation marker; comparing the cell culture samples for relative levels of activation response; assessing whether a shift in the grid position of the white blood cells in the cell culture sample containing the antigen, compared to the other cell culture sample, demonstrates that the white blood cells have been activated in response to antigen recognition, and thus that the antigen is immunologically recognizable.

### BRIEF DESCRIPTION OF DRAWINGS

FIG. 1 shows a black-and-white photograph of an electrophoresis gel illustrating the molecular weights of various lacteal secretions.

FIG. 2 is a chart providing mass spectrometry analytical data for a sample from a normal, uninfused lacteal secretion (i.e., normal milk).

FIG. 3 is a chart providing mass spectrometry analytical data for a sample from a traditionally infused lacteal secretion.

FIG. 4 is a chart providing mass spectrometry analytical data for a sample from a gun-inoculated lacteal secretion.

FIG. 5 is a chart showing immune activation in Group I mice in Example 5 resulting from administration of a Bioengineered Lacteal Complex (“BLC”) according to the present invention.

FIGS. 6 to 9 are each charts reflecting in vitro antimicrobial activity of a BLC according to the present invention prepared and initially administered to Group I mice in Example 5.

### DETAILED DESCRIPTION OF INVENTION

We have developed immunodynamic complexes that, in an embodiment, are both antimicrobial and immunoactive. By immunoactive, we mean, in an embodiment, that they are capable of modulating, stimulating and repairing the immune system. Moreover, in an embodiment, we use the term “immunoactive” to mean an immunodynamic complex that is capable of maintaining, supporting and enhancing the structure and function of the immune system. In addition, we have developed a method for preparing and using such complexes. As to the method for preparing such complexes, our focused discussion here is on the preparation of such a complex from lacteal secretions derived from ungulates. However, it should be understood that this invention is not limited to the production of such complexes only from lacteal secretions. Rather, we envision that such complexes also could be produced by a synthetic method. However, at this stage, the most cost-effective manner to prepare such a complex is by bioengineering specific lacteal secretions from ungulates.

In terms of definitions used in this application, we use the term Refined Lacteal Complex (“RLC”) to mean a lacteal secretion from an ungulate that has been refined in any manner. Moreover, we use the term Bioengineered Lacteal Complex (“BLC”) to mean a RLC that has been derived from an ungulate that has been gun-inoculated with an antigen/cytokine mixture into the udder in order to produce specific immunodynamic complexes. Additionally, as noted above, any synthetically produced immunodynamic complex that has similar properties to an ungulate-derived BLC could also be termed a BLC.

### General Overview

Our invention comprises at least three embodiments, including: 1. manufacturing a bioengineered lacteal complex (a BLC); 2. assaying the manufactured products for viability, consistency, potency and purity; and 3. utilizing a BLC of the present invention as an antimicrobial and immunoactive composition.

We have found that, in an embodiment, a novel immunodynamic complex comprising a fraction of proteins and peptides of about 100 kD molecular weight and less (i.e. a BLC) can be harvested from lacteal secretions of ungulates, such as cows, that have had inoculated into their udder a specially prepared antigen/cytokine preparation or mixture. In one embodiment, the antigen/cytokine mixture can be inoculated into an ungulate, such as a cow, with an inoculation gun into at least one quarter of the udder. We believe that the resulting BLC can provide a number of benefits. First, in an embodiment, we believe that it can provide a direct antimicrobial effect against a specific microorganism or class of microorganisms as a treatment for a condition or conditions resulting from a patient's pathogenic exposure and/or infection. Moreover, because of a BLC's antimicrobial precision, we believe that a BLC, in an embodiment, does not have an antimicrobial effect on non-pathogenic microorganisms. Second, in another embodiment, we believe that a BLC is immunoactive in that it is capable of immune system stimulation by causing immune modulation and repair in an immunocompromised patient. Third, in yet another embodiment, we believe that a BLC's immunoactive characteristics render it capable of creating a passive immunization response (i.e., a vaccination-like effect), including being capable of preventing disease symptoms after known or unknown exposure to an organism or a class of specific pathogens. Fourth, in still another embodiment, in an effective amount or when two or more BLCs are blended, we believe that the resulting product can maintain, support and enhance the structure and function of the immune system in a healthy subject.

According to our research, we have found that a BLC of the present invention contains molecules that do not appear in material from animals that have not been gun-inoculated with a novel antigen/cytokine mixture of the kind that we have developed and that is described further below. Some of the molecules that we have identified in our novel BLC include defensins, granulysins and transfer factors, which are specific to the pathogen for which the animal was injected, as well as lactoferrin. Moreover, a BLC of the present invention contains many cofactor molecules that we have not yet specifically identified but that we know are present based on the testing that we have conducted using various laboratory techniques, including electrophoresis, mass spectrometry and protein sequencing testing, which we also discuss later.

We also have developed a special assay to validate the quality of a BLC of the present invention by detecting the presence of defensins, granulysins, transfer factors and lactoferrin in the BLC. These molecules can be identified by electrophoresis and mass spectrometry in conjunction with N-terminal amino acid sequencing. Moreover, we have developed a method for validating the quality of the antigen used in the antigen/cytokine mixture. These assays have been developed as important methods for controlling the quality of a BLC of the present invention.

### An Embodiment of a Method of Making the Immunodynamic Complex

An embodiment of a method of making the immunodynamic complex of the present of invention is described here. The various steps are also described in Examples 1 and 2 provided below. Initially, the antigen/cytokine mixture can be prepared. An exemplary method of making the antigen/cytokine mixture is provided in Example 1. First, one may combine an antigen with an assortment of cytokines. In this specification, we envision that the term “antigen” refers to any antigen-like material that includes, but is not limited to cells, antigens or cellular fragments from bacteria, viruses, mycobacteria, yeasts, mold, spirochetes, cancer cells, allergens and other pathogens. Moreover, we envision that various assortments of cytokines could be used in the antigen/cytokine mixture. We use the term “cytokine” as a generic name for a diverse group of soluble proteins and peptide adjuvants that act as immune regulators at nanomolar to picomolar concentrations and that, either under normal or pathological conditions, modulate the functional activities of individual cells and tissues. In an embodiment, the following cytokines may be used in the antigen/cytokine mixture: alpha-TNF, G-INF, IL-8, and GM-CSF.

An embodiment of an antigen/cytokine mixture for inoculation into an ungulate, such as a cow might contain: (a) an amount of a 10% solution of fetal calf serum and phosphate buffered saline, or a 1% solution of bovine albumin and phosphate buffered saline; (b) an amount of an antigen; (c) an amount of Alpha-TNF; (d) an amount of G-INF; (e) an amount of IL-8; and (f) an amount of 2 cc GM-CSF. Approximate ranges for each of these components at a given concentration, for example as discussed in Example 1, are set forth in the chart provided below:

| | Broad Range | Medium Range | Narrow Range |
|---|---|---|---|
| Fetal Calf | 0.1 cc to 20 cc | 1 cc to 10 cc | 3 cc to 7 cc |
| Serum or | | | |
| Bovine | | | |
| Albumin | | | |
| solution | | | |
| Antigen | 0.1 cc to 20 cc | 1 cc to 10 cc | 3 cc to 7 cc |
| Alpha-TNF | 0.001 cc to 2 cc | 0.01 cc to 1 cc | 0.05 cc to 0.4 cc |
| G-INF | 0.001 cc to 2 cc | 0.01 cc to 1 cc | 0.05 cc to 0.4 cc |
| IL-8 | 0.001 cc to 2 cc | 0.01 cc to 1 cc | 0.05 cc to 0.4 cc |
| GM-CSF | 0.001 cc to 2 cc | 0.01 cc to 1 cc | 0.05 cc to 0.4 cc |

These approximate ranges for the antigen/cytokine mixture are for one cow for one inoculation session (e.g., one inoculation dose into each of four quarters of an udder). It should be noted that the fetal calf serum solution or bovine albumin solution can be made with phosphate buffered saline (i.e., an isotonic 0.90% weight per volume solution) and are used in this instance as carriers for cytokines.

Once the antigen/cytokine mixture is prepared, this mixture is introduced into the udder of an ungulate, such as a cow. The mixture may be introduced using a CO₂ powered inoculation gun. This is accomplished by loading the inoculation gun with the desired antigen/cytokine mixture and injecting the mixture into the udder of the ungulate. For example, the MED-E-JET™ injection gun could be used. The following approximate gun pressure and gun volume ranges might be appropriate in an embodiment of our inventive approach to producing a BLC:

| | | |
|---|---|---|
| Gun pressure setting | Broad | 250 psi to 800 psi |
| ranges: | Medium | 350 psi to 750 psi |
| | Narrow | 400 psi to 650 psi |
| Gun volume ranges: | Broad | 0.1 cc to 2 cc |
| | Medium | 0.5 cc to 1.5 cc |
| | Narrow | 0.7 cc to 1.0 cc |

Additional details as to our preferred method of using the inoculation gun are provided in Example 2. Also, in an embodiment, the antigen/cytokine mixture is introduced into the udder of the ungulate any time post partum, that is, in the milking stage.

Thereafter, starting about 2 days following the inoculation, the ungulate can be milked, and the milk product (i.e., lacteal secretion) that is obtained can then be processed as described in more detail in Example 2 to produce, for example, a liquid product or a dried product. A dried product according to the present invention can be prepared, for example, by going through the following steps: collection, freezing, thawing, pasteurizing, casein removal, freezing and thawing again, microfiltrating, conducting reverse osmosis, freezing and pasteurized again if appropriate, and spray drying. On the other hand, a liquid extract can be prepared, for example, by going through the following steps: collection, freezing, thawing, pasteurizing, casein removal, freezing and thawing again, blending with other products if necessary, ultra filtrating, adding a desired preservative if necessary, and packaging aseptically.

In an embodiment, during processing, the milk product (i.e. lacteal secretion) is filtered so as to create a BLC containing molecules that are less than about 100,000 Daltons (D) or 100 kD. For example, an ultrafiltration process may be used to remove large protein molecules and contaminates from the whey permeate that contains the desired immunodynamic molecules having molecular weight of less than about 100 kD. The filtrate containing material of less than about 100 kD should contain a collection of antigen specific molecules that include but are not limited to granulysins, defensins and transfer factors. It is this filtrate that can be referred to as a BLC. In addition to the antigen-specific molecules in the less-than-about-100 kD material from the ungulate gun-inoculated with the antigen/cytokine mixture, a BLC of the present invention may contain numerous factors that may also be present in colostrum, milk and whey that could also be beneficial.

In an embodiment, the resulting BLCs can be blended to produce a product that is capable of supporting the structure and function of a healthy immune system.

### The Antigen/Cytokine Mixture

Example 1 provides a detailed description of how to prepare an antigen/cytokine mixture that is used in an embodiment of our invention. Generally speaking, the following components may be present in the antigen/cytokine mixture: an antigen, and cytokines such as alpha-TNF, G-INF, IL-8, and GM-CSF. However, we envision that other combinations of cytokines may also be useful to prepare an antigen/cytokine mixture according to the present invention. The nature of each of alpha-TNF, G-INF, IL-8, and GM-CSF is described further below, based on current immunological understanding of such cytokines. However the present invention should not be seen as limited by such theory of cytokine activity.

Alpha-TNF is secreted by macrophages, monocytes, neutrophils, T-cells, NK-cells following their stimulation by bacterial lipopolysaccharides. Cells expressing CD4 secrete alpha-TNF while CD8+ cells secrete little or no alpha-TNF. Alpha-TNF is also secreted by stimulated and unstimulated peripheral neutrophilic granulocytes, as well as a number of transformed cell lines, astrocytes, microglial, smooth muscle cells, and fibroblasts. The 26 kD form of alpha-TNF is found predominantly on monocytes and T-cells after cell activation. It is also biologically active and mediates cell destruction by direct cell-to-cell contacts. Alpha-TNF mediates part of the cell-mediated immunity against obligate and facultative bacteria and parasites. It can confer protection for example, against Listeria monocytogenes infections, and anti-TNF antibodies weaken the ability of mice to cope with these infections. Alpha-TNF has an immunomodulatory effect on various immune effector cells, including neutrophils, macrophages, and T-cells.

INF-gamma (i.e., G-INF) is produced mainly by T-cells and natural killer cells activated by antigens, mitogens, or alloantigens. It is produced by lymphocytes expressing the surface antigens CD4 and CD8. INF-gamma has antiviral and antiparasitic activities and also inhibits the proliferation of a number of normal and transformed cells. INF-gamma synergises with TNF-alpha and TNF-beta inhibiting the proliferation of various cell types. The growth inhibitory activities of INF-gamma are more pronounced than those of the other interferons. However, the main biological activity of INF-gamma appears to be immunomodulatory in contrast to the other interferons, which are mainly antiviral.

INF-gamma also stimulates the expression of antigens on cell surfaces, the expression of CD4 in T-helper cells, and the expression of high-affinity receptors for IgG in myeloid cell lines, neutrophils, and monocytes. In monocytes and macrophages INF-gamma induces the secretion of TNF-alpha and the transcription of genes encoding G-CSF and M-CSF. In macrophages INF-gamma stimulates the release of reactive oxygen species.

The IL-8 receptor is a dimeric glycoprotein consisting of a 59 kD and a 67 kD subunit. It has been given the name CD128. It is expressed in many different cell types including those not responding to IL-8. The receptor density is approximately 20000/cell in neutrophils and approximately 300/cell in T-lymphocytes. IL-8 is produced by stimulated monocytes but not by tissue macrophages and T-lymphocytes. IL-8 is produced also by macrophages, fibroblasts, endothelial cells, keratinocytes, melanocytes, hepatocytes, chondrocytes, and a number of tumor cell lines. The activities of IL-8 are not species-specific. Human IL-8 is also active in rodent and rabbit cells. The biological activities of IL-8 resemble those of a related protein, NAP-2 (neutrophil-activating peptide-2). IL-8 differs from other cytokines in its ability to specifically activate neutrophil granulocytes.

GM-CSF protein is secreted together with other factors by T-cells and macrophages following cell activation by antigens or mitogens. Approximately 90 percent of the secreted colony stimulating activities are due GM-CSF. GM-CSF can be associated with the extracellular matrix of cells as a complex with heparin sulfate proteoglycans. This allows storage of the factor in a biologically inactive form. The exact mechanism by which the factor is eventually released from these depots is not known. GM-CSF can be expressed also as an integral membrane protein. The membrane-bound or matrix-associated forms of the factor can interact with receptors on other nearby cells. This effectively establishes cell-to-cell contacts and may induce biological activities also in juxtaposed cells. GM-CSF stimulates the proliferation and differentiation of neutrophilic, eosinophilic, and monocytic lineages. It also functionally activates the corresponding mature forms, enhancing, for example, the expression of certain cell surface adhesion proteins (CD-11A, CD-11C).

As to the antigen, we have developed a novel method by which to control the quality of the antigen that we prepare and use in the antigen/cytokine mixture. This quality control method is discussed further Example 1.

### Gun Inoculation

We have discovered that gun-inoculation application of antigens does not tend to adversely affect somatic cell count and, therefore, does not cause an inflammatory response in the udder. If there is no inflammatory reaction in the udder, the response to antigens in the udder can be channeled to produce immunodynamic complexes with its full resources, as there is no energy allocated to combat a local inflammation. We believe this may contribute to more immunodynamic complexes being produced in a BLC of the present invention. Moreover, bactericidal testing described in Example 3 below, shows that lacteal material from cows given a gun inoculation with our novel antigen/cytokine mixture (i.e., a BLC) is on the order of 2 to 2.5 times more bactericidal than lacteal material from cows given a traditional infusion with a traditional antigen mixture. Consequently, in an embodiment, our novel antigen/cytokine mixture may be administered to an ungulate by way of gun inoculation, as opposed to traditional infusion.

In addition, traditional infusion, as practiced in the industry, is generally done only at pre-calving and the lacteal secretions from pre-calving animals contain sufficient quantities of immunoactive molecules for only 8 to 10 weeks post partum. Gun inoculation, on the other hand, can be done throughout the milking period, thereby increasing the productivity 8 to 10 times. Also, if gun inoculation is applied post-partum, the cow (or other ungulate) is less biochemically and immunologically stressed after calving. This allows the udder of the cow (or other ungulate) to be even more effective in producing immunodynamic molecules and is a healthier situation for the animal.

### Various Immunodynamic Molecules in a BLC

Electrophoresis reveals that a BLC of the present invention surprisingly is different from normal lacteal secretions (i.e., normal milk) and is different from traditionally infused material obtained from lacteal secretions. As suggested earlier, there are two variables that are changing between traditional lacteal secretions and lacteal secretions of the present invention. First, instead of using an antigen mixture, our approach uses an antigen/cytokine mixture. Second, our approach may use an inoculation gun as opposed to an infusion process.

We have identified many unique components in a BLC of the present invention using various techniques as described below. Moreover, by this analysis, we have also determined that the material of the present invention contains many cofactor molecules that we have not yet specifically identified but that we know are present.

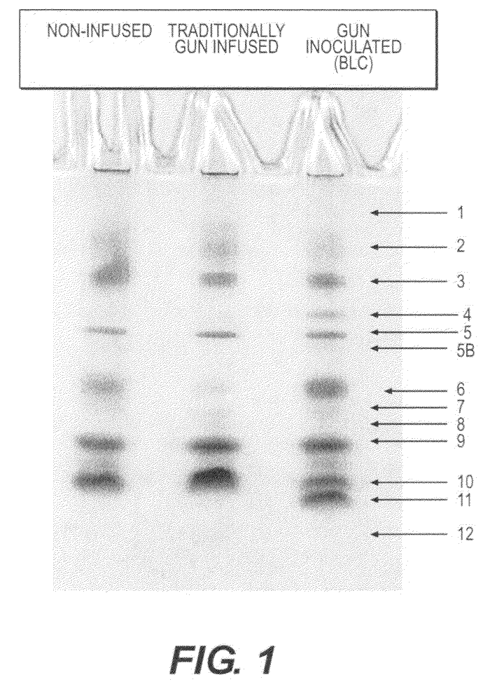

For example, using an electrophoresis gel technique, we can separate the molecules from a BLC of the present invention into bands by molecular weight. The molecular bands of a BLC are significantly different from bands in material from non-inoculated cows and from traditionally infused cows. Electrophoresis shows shifts of molecular material in the less than 100 kD range, and a particularly dramatic increase of molecular material in the molecular range of defensins, granulysins and transfer factors. The observation of a molecular shift is supported by the appearance of new bands without an apparent increase in the overall quantity of molecules. This indicates that the production of molecules are shifted from one molecular range to another to produce molecules that (when tested) are specific to the inoculated antigen. A more detailed discussion of our electrophoresis gel analysis is provided below in the context of detailed description of FIG. 1.

Moreover, mass spectrometry analysis of a BLC of the present invention material as compared to a normal lacteal secretion (i.e., normal milk) or a lacteal secretion obtained by a traditional infusion technique shows that there are many molecules in each electrophoresis band of such a BLC that differs in quantity and quality from such other lacteal secretions. When further analyzed, the molecular weight of specific molecules of a BLC to have been found to correspond to many known immune system cytokines and co-factors.

Protein sequencing has confirmed the presence of more than one protein in each band. Consequently, it is difficult to identify specific proteins from a gel electrophoresis alone. By using the protein sequencing technique, we have validated the presence of over 100 molecules in a BLC of the present invention that support the modulating, stimulating, immunizating and bactericidal effect of the material. Our analysis in this regard involved electrophoresing a sample of a BLC and then transferring the separated sample to a PVDF membrane. The main bands identified in the gel electrophoresis were cut out of the membrane and then sequenced. By studying the results of the sequencing, we were able to find that there are several proteins/peptides present within each band.

FIG. 1 readily shows the “molecular shift” in electrophoresis gels that occurs when comparing a normal lacteal secretion, a lacteal secretion obtained from traditional infusion, and our novel BLC, obtained, for example, from a gun-inoculated lacteal secretion. By “molecular shift”, we mean that not only do we find molecules in our inventive BLC not generally present in traditional lacteal secretions or at least not in significant amounts, but also, that there are larger amounts of such molecules in the less than 100 kD range in a BLC of the present invention. In particular, we find that there are more granulysins, transfer factors and defensins in a BLC of the present invention. This is certainly the case with the preferred form of our inventive BLC depicted in FIG. 1.

Specifically, FIG. 1 shows an electrophoresis gel with 3 lanes loaded with the following samples, from left to right: 1. an uninfused lacteal secretion (i.e. normal milk); 2. a lacteal secretion obtained from a traditional teat infusion method with an antigen mixture; and 3. a lacteal secretion obtained from a gun inoculation method of the present invention using an antigen/cytokine mix (i.e., a BLC). The gel electrophoresis depicted in FIG. 1 compares the polypeptide content for polypeptides under 100 kD. We have identified 12 different main bands of protein/peptide material in the BLC sample. These bands are labeled in FIG. 1. Two different molecular ladders were used to establish the approximate molecular weights of different bands. They were run using the electrophoresis protocol, then confirmed with biological activity testing. Based upon biological activity and molecular weight, we have identified certain components in various bands of our separated gun inoculated sample, as described below:
Band 1: IgM Antibody Fragment at 95-130 kD (In embodiments of a BLC of the present invention, this may be filtered out)
Band 2: Lactoferrin at about 90 kD
Band 11: Granulysin at about 9 kD
Band 12: Defensins and Transfer Factors at about 5 kD
   The identity of proteins in Bands 2, 11 and 12 were further confirmed when tested for antimicrobial effect, as discussed in detail in Example 3.

Although Band 12 cannot be readily seen in FIG. 1, we are able to see this band under a magnifying glass. Subsequent testing of the material in Band 12 involving mass spectrometry and protein sequencing testing as well as the defensins and transfer factor antimicrobial testing described in Example 3 below, all confirm that Band 12 contains defensins and transfer factors.

A molecular shift can be demonstrated in the bands at levels 1, 2, 3, 4, 5B, 6, 7, 9, 10, 11 and 12. Bands begin to shift in the material from gun inoculated cows by creating new bands, more dense bands and wider bands. Specifically, band 4 is only barely visible in columns 1 and 2 but is clearly visible in column 3. Although not entirely apparent from FIG. 1, when viewed under a magnifying glass, band 5B is only seen in the gun inoculated sample in column 3. Band 6 is darkest in the gun inoculated sample in column 3. Although also not entirely apparent from FIG. 1, when viewed under a magnifying glass, Band 8 is only seen in the traditionally infused sample in column 2. Band 11 is only seen in the gun inoculated sample in column 3. Again, although not entirely apparent from FIG. 1, when viewed under a magnifying glass, Band 12 is seen in both column 2 and column 3. Bands that can be identified with a magnifying glass also have been verified by mass spectrometry.

Although we have not yet identified all of the molecules presented in our inventive gun inoculated lacteal secretion, we believe that many of the unidentified molecules are support and modulatory molecules, including mini-cytokines. We believe that mini-cytokines, in particular, are present because of the biologically active granulysins, which we describe in more detail below. Modulatory molecules are presumed to be present because of the transfer factor effect from tissue cell culture testing, which is also discussed in more detail later and illustrated in Part 7 of Example 3.

Consequently, when comparing molecular bands from a source of refined lacteal secretions from animals that were gun-inoculated with refined lacteal secretions from traditional antigen infusion and lacteal secretions from non-infused animals, we have observed an appreciable “molecular shift.” We have found that, when an antigen/cytokine mixture is gun-inoculated into the udder of an ungulate, such as a cow, the ungulate's udder and the ungulate's immune system respond to that antigen/cytokine mixture by producing immunodynamic molecules that cause the production of molecules to “shift” from what it was producing based upon its exposure to antigens in the environment, to produce antigen-specific molecules in response to the introduced antigen.

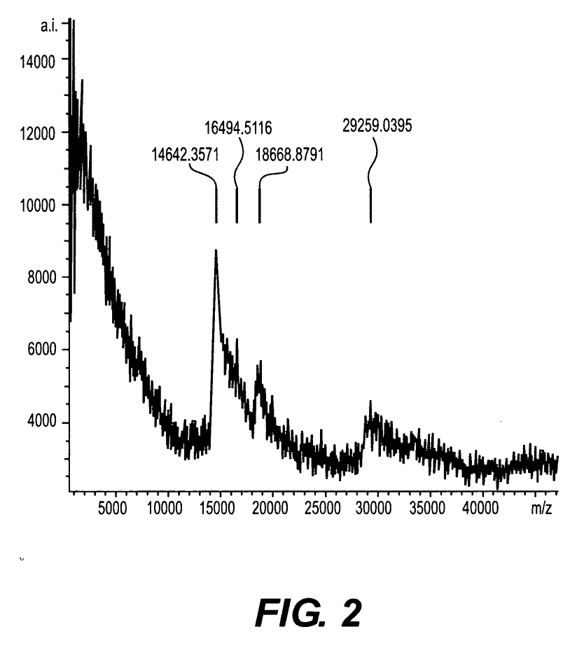

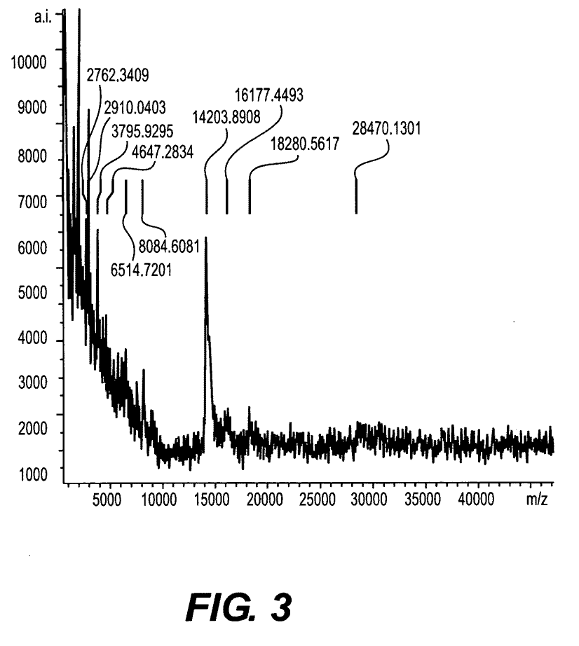

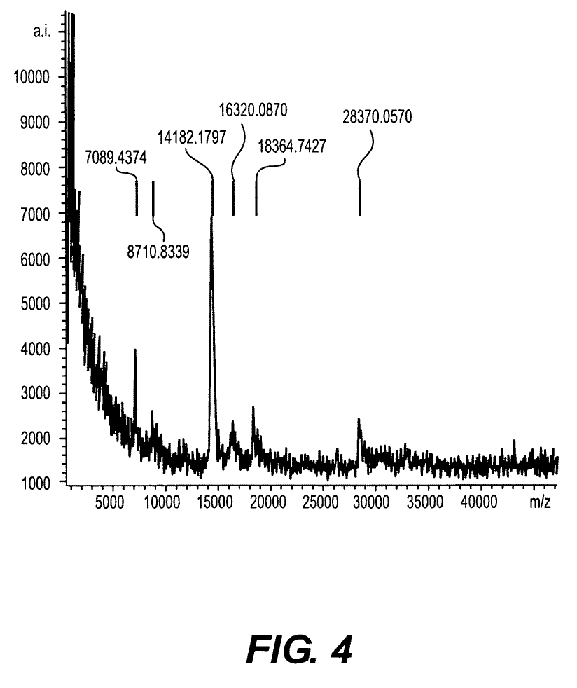

FIGS. 2, 3 and 4 show data from mass spectrometry analyses conducted on three different samples. The samples used were various lacteal secretions that were processed but not separated by molecular weight. FIG. 2 is a chart providing mass spectrometry analytical data for a sample from a normal, uninfused lacteal secretion (i.e., normal milk). FIG. 3 is a chart providing mass spectrometry analytical data for a sample from a traditionally infused lacteal secretion. Among other things, FIG. 3 shows an increased production of low molecular weight small molecules. FIG. 4 is a chart providing mass spectrometry analytical data for a sample from a gun-inoculated lacteal secretion of the present invention (i.e., a BLC). Interestingly, the mass spectrometry curves shown in FIGS. 3 and 4 are different in important respects. For example, as compared to the chart provided at FIG. 3, the chart at FIG. 4 includes the appearance of a significant granulysin band at about 8710 Daltons (or about 8.7 kD) (i.e. a molecular weight between about 8500 kD and about 9500 kD (or between about 8.5 kD and about 9.5 kD)). Consequently, these mass spectrometry results yielded more detailed information about the number of molecules present at different molecular weights. Some of the molecules in high concentration are noted by their molecular weights on the charts. This information mirrors both the banding that is demonstrated by gel electrophoresis analysis and the number of molecules seen by amino acid sequencing.

Thus, a BLC of an embodiment of the present invention can be separated by electrophoresis into more than 12 different bands containing more than one hundred molecules that can interact to generate the various immunological effects that the BLC demonstrates. Of these myriad molecules, some of the ones that have been identified here are granulysins, transfer factors, defensins and lactoferrin. These molecules have been clearly identified by way of an amino acid-sequencing assay. The granulysins, defensins and lactoferrin demonstrate an antimicrobial effect and transfer factors demonstrates a transfer effect, as shown in Example 3. Also, as will be discussed later, we have found that our inventive BLC can be characterized by assaying for the presence of granulysins, transfer factor and defensins.

Moreover, in an embodiment of a BLC of the present invention, the molecules in such a BLC, such as granulysins and defensins can demonstrate an antimicrobial effect as against the antigen used in the antigen/cytokine mixture. In particular, in Example 3, we provide evidence that some of the molecules in a BLC of the present invention are specific for killing the bacteria that were used in the antigen/cytokine mixture. That is, there is a specificity of the molecules against the infused pathogen. Our research also has shown that, if the antigen/cytokine mixture includes a streptococcus, for example, there may also be some cross reactivity against other gram positive organisms like staphylococcus.

In addition, Part 7 of Example 3 illustrates that transfer factors in a BLC of the present invention are capable of passively transferring immunity from one organism to another by stimulating cell mediated immunity, such that the organism acquiring such immunity can, for example, launch its own antimicrobial attack by producing antigen-specific defensins.

To better understand the biological activity of granulysins, defensins, lactoferrin, mini-cytokines and transfer factors in our inventive BLC, more information about each of these molecules is provided below. Each of these molecules has a mass of less than or equal to about 100 kD. This discussion is based on current immunological understanding. However, the present invention should not be seen as limited by such theory.

Accordingly to our current understanding, the actions of defensins are varied and multidimensional. On direct contact, defensins are bactericidal and attract macrophages, and to an extent modulate local inflammation. In addition, their presence is reflective of a multitude of “up stream” immunological activity.

Defensins are small basic unglycosylated proteins of 29-34 amino acids. Multiple forms of defensins exist in humans and it has been shown that there are 9 defensins in humans, 6-alpha-defensins and 3-beta-defensins. Alpha defensins at about 3 kD are made and stored in neutrophils. These molecules up-regulate the expression of TNF-α that activates T4 cells and also up-regulates the expression of IL-1, which modulates inflammation. There are 5 kD beta-defensins that are involved in the chemotactic recruitment of lymphocytes and dendritic cells and thus serve as one bridge between innate and adaptive immunity.

In micromolar concentrations, defensins are bactericidal for a variety of gram-negative and gram-positive microorganisms, mycobacteria, and yeasts, and some enveloped viruses. Defensins also possess a nonspecific cytotoxic activity against a wide range of normal and malignant cells, including cells that are resistant to TNF-alpha and NKCF (natural killer cytotoxic factor). For example, the existence of enteric defensins suggests that these peptides contribute to the antimicrobial barrier function of the small bowel mucosa, protecting the small intestine from bacterial overgrowth by autochthonous flora and from invasion by potential pathogens that cause infection via the peroral route, such as Listeria monocytogenes and Salmonella species.

At about 9 kD are granulysins. Accordingly to our current understanding, granulysins are induced by IL-15, which is found in many body cell types and are made by LAK (lymphokine activated killer) cells and NK cells. Granulysins are a T-cell activation marker that has been shown to be effective as an antimicrobial agent. Granulysins have been identified as an effector molecule co-localized with perforin in the cytotoxic granules of cytotoxic T lymphocytes and natural killer (NK) cells. It is used in vitro and in vivo to reduce the population of viable cells in a microbial population.

Due to the tertiary molecular folding of defensins and granulysins, it is difficult at this time to synthesize defensins and granulysins that are biologically effective. Also, as there are a number of smaller support and co-factor molecules that improve the functioning of these two molecules, using purified extracts of defensins and granulysins to date has proved to be of minimal value. In this regard, we note U.S. Pat. No. 6,485,928, which suggests that granulysins on their own, are effective as an antimicrobial agent. However, our research does not support this finding. Consequently, we believe that our inventive approach for producing an immunodynamic complex is not only a cost-effective process, but also an approach that, in an embodiment, can efficiently produce therapeutically-effective compositions.

Lactoferrin is an iron-binding glycoprotein of the transferring family, first isolated from milk but also found in most exocrine secretions as well as the secondary granules of neutrophils. The many reports on its antimicrobial and inflammatory activity in vitro identify lactoferrin as important in host defense against infection and excessive inflammation. Lactoferrin exerts its effects on glandular epithelia, secretions, mucosal surfaces as well as in the interstitium and vascular compartments where it has been postulated to participate in iron metabolism, disease defense and modulation of inflammatory and immune responses.

Minicytokines is a term used to denote peptide factors that are not normally classified as cytokines or growth factors due to their small molecular weights. For example, these substances can operate as cytokines and show all of the major properties of cytokines. They can be produced by immune cells and they can exert their actions in panacrine, autocrine and endocrine ways. Moreover, they often show functional redundancy, pleiotrophy and effects that are both dose- and time-dependent.

Transfer factors are small protein molecules that can passively transfer immunity from one organism to another by stimulating cell mediated immunity. They have molecular weights of approximately 5 kD and appear to be composed entirely of amino acids (although recent papers describe a nucleo-peptide molecular complex). According to our understanding, these molecules “educate a recipient's immune system” by transferring antigen specific targeting information. Most interactions of the cell mediated immunological (“CMI”) response are initiated through the activity of the T-cell lymphocytes. They are a special type of white blood cell that receives “training” mostly in the thymus. The thymus gland swells in size from birth to puberty and then begins to shrink through a process called involution. As the clones of T-cells mature in the thymus they learn to recognize specific antigens. Consequently, transfer factors can be considered to be immunoactive molecules.

In the process of sequencing the molecules discussed above, one additional sequence has also shown prominently, that being albumin. Albumin appeared in more than one band. We interpret this as meaning that some of the immuno-active complexes, such as those discussed above, are bound to albumin. According to our understanding, albumin is a transporter molecule for a number of different cytokines. The fact that albumin is present in BLCs of the present invention suggests that the cytokines are very active, since the cytokines are sticking to other molecules. Cytokines and minicytokines are active on the surface of the cell membranes and inter-cellularly. The albumin can then transport them to the cell membranes where they attach to specific receptor cell sites and membrane pumps, which introduces the molecules into the cells. In the modulating effect discussed later, primary and secondary messenger molecules are induced inside the cell to create new receptor sites and reactivate a pathway. This happens when cytokines and mini-cytokines, are attached to the cell receptor sites. The albumin can carry the cytokine to the cell membrane and the cytokine can penetrate into the cell and induce a whole cascade of secondary molecules that results in the production of new cell membrane receptor sites and physically reactivate a cytokine pathway.

### Assay for Validating the Content of a BLC

The present invention includes methods for validating the creation of select molecules in a BLC of the present invention. The immunodynamic molecules produced for the ungulate upon administration into the udder of an antigen/cytokine mixture of the present invention are identifiable by an assay that we have specifically developed for this purpose. These immunodynamic molecules are antigen-specific to the antigen used in the antigen/cytokine mixture. Consequently, such molecules are predictable and specific to the antigen/cytokine mixture that is used and can be measured. This measurement can identify the molecules that are present by quantity and quality. In an embodiment, prior to using a batch of a BLC for therapeutic or other purposes, the batch can be assayed. Our assay utilizes gel electrophoresis and UV protein spectrometry. These techniques are generally known to those of ordinary skill in the art and thus are not described in detail here.

In an embodiment, our assay method involves isolating molecular bands of defensins, granulysins, transfer factors, lactoferrin and other specialized molecules in the less than about 100 kD material that comprises a BLC. Thereafter, we can measure concentration of these select molecules. The purpose of this assay is to monitor the “molecular shift” that occurs when comparing: (1) the lacteal secretion prepared upon administration of an antigen/cytokine mixture to an ungulate (i.e., a BLC); with (2) a lacteal secretion prepared by the traditional method of antigen infusion; and (3) a normal lacteal secretion (i.e. normal milk). This shift can be quantified with reverse electrophoresis and densitometry of the gel. In particular, we find that there are more granulysins, transfer factors and defensins in a BLC according to the present invention than there is in a lacteal secretion obtained by a traditional infusion method.

Subsequently, we can then take a BLC of the present invention and test its antimicrobial effect. This helps demonstrate that our gun inoculation process was successful in producing an effective BLC, and can be used for quality assurance. Example 3 provides additional explanation as to microbiological testing conducted on the refined lacteal complex produced according to the methods described in Examples 1 and 2.

### Methods of Using a BLC

As suggested earlier, we envision that a BLC according to the present invention could be used in a number of different embodiments. At the outset, it should be noted that we envision a BLC of the present invention being useful for any living organism, including humans and animals. In one embodiment, we believe that a BLC could be used as an antimicrobial composition to treat an acute infection in a patient. The manner in which we envision that such treatment might progress is described in Part 1 of Example 4. In a second embodiment, we believe that a BLC could be used to treat a patient with a chronic infection to help restore immune function. The manner in which we envision that such treatment might progress is described in Part 2 of Example 4. In a third embodiment, we believe that a BLC could impart an immunization (or a vaccination-type effect) against a specific antigen. The manner in which we envision that such treatment might progress is described in Part 3 of Example 4. In yet a fourth embodiment, we believe that a BLC or a blend of two or more BLCs could be used in amounts effective to support the structure and function of the immune system. An example of how we envision such use in a generally healthy individual is described in Part 4 of Example 4.

A BLC of the present invention can be provided in a liquid form or powder form. Such a BLC can be utilized as a liquid that is processed by asceptical bottling for direct consumption. Alternatively, the BLC can be further processed by spray drying to a powder form.

In an embodiment, the refined, final form of the BLC can then be further processed by known methods to produce a pharmaceutically acceptable composition. In certain instances, this may require combining the BLC together with an acceptable pharmaceutical carrier, whether that carrier is in a liquid or solid format. For example, the BLC can be further processed so as to be administered in any suitable liquid or powder form, such as by pill, capsule, liquid, liquid gargle, lollipop, food additive, injection, transdermal patch, cream, intravenous solution, ear/eye/vaginal drops or spray, suppository, liposomes, gun inoculation or any other method of application and ingestion.

In yet another embodiment, a dietary supplement containing one or a blend of BLCs might be prepared for oral ingestion by healthy subjects in amounts effective to support, maintain and/or enhance the structure and function of the immune system in the following forms: tablet, capsule, powder, softgel, gelcap, and liquid form.

In an embodiment, the following chart provides possible approximate dosage ranges for a BLC administered via various different routes.

### Powder—Oral and Transdermal (i.e., Topical)

Broad Range—10 mg to 2500 mg
Medium Range—50 mg to 2000 mg
Narrow Range—100 mg to 1500 mg

### Liquid—Oral, Intravenous, Inter-peritoneal & Subcutaneous

Broad Range—1 cc to 20 cc
Medium Range—5 cc to 15 cc
Narrow Range—7 cc to 10 cc

Intramuscular, Injection, Gun inoculation
Broad Range—1 cc to 10 cc
Medium Range—1.5 cc to 7 cc
Narrow Range—2 cc-to 4 cc

Transdermal
Broad Range—0.1 cc to 180 cc
Medium Range—0.5 cc to 60 cc
Narrow Range—1 cc to 5 cc

Intranasal
Broad Range—0.1 cc to 10 cc
Medium Range—0.2 cc to 5 cc
Narrow Range—0.5 cc to 1 cc

The transdermal dosages address necessary amounts for transdermal application for burn patients for application with spray application. Although spraying is likely the most appropriate mode of transdermal application for infection control, another transdermal application option might be submersion. Submersion would require on the order of 50 gallons rather than 40 cc.

In an embodiment, a patient might be administered a daily dosing of about 5 cc or less of liquid refined lacteal complex containing about 20% of solids, 3 times daily, or about 1000 mg or less of dried material, 1 time daily.

### Immunological Theory Behind the Activity of a BLC

While we do not wish to be bound by theory, the following explanation is included to provide our current understanding as to how a BLC according to the present invention might function in various embodiments of the present invention, as an immunodynamic composition.

As has been discussed previously, a BLC of the present invention acts, in an embodiment, as an antimicrobial composition. In this regard, we have discovered that a BLC is capable of acting as a selective killing agent against pathogenic strains of bacteria. For example, we have found that a BLC can kill a pathogenic strain of streptococcus but will not harm a non-pathogenic strain of streptococcus. Laboratory testing confirming this finding is provided in Example 3. We would expect that our laboratory test results would be further confirmed by in vivo evidence in that a patient to whom a BLC is administered would not have a sterile gut.

In an embodiment, according to our understanding, a fraction of a BLC of the present invention is immunoactive. For example, a BLC in a cell culture demonstrates a transfer factor effect or a molecular communication effect that is caused by transfer factors. This effect was mentioned previously, and is illustrated in Part 7 of Example 3. That is, we have validated this effect in vitro by putting in a very small amount of a fraction of a BLC of the present invention (i.e., as found in Band 12 on an electophoresis gel) into a cell culture and letting the leucocytes grow for 24 hours. We have found that the material that comes out of cell culture, the supernatant, then becomes specifically antimicrobial. When tested, the material is as bactericidal as the original material even though there is essentially none of the original material in what was taken from the supernatant. We believe that this happens because the original material induces the production, for example of defensins in the prepared culture, which in part is a demonstration of a transfer factor effect. We believe that this indicates that a BLC contains molecules that can communicate between different cells of the immune system to produce something that such cells were not producing before. This is an active process (i.e., an immunoactive process) rather that a passive process that we believe demonstrates that a BLC may be inducing white blood cells to produce something that they were not previously producing.

### 1. The Bridging and Spreading Effects

We believe that a BLC of the present invention could, in an embodiment, also act as an immunoactive complex in other ways. In particular, we believe that molecules in a BLC of the present invention, and particularly those below about 10 kD (e.g., such as defensins, granulysins, transfer factors and other nucleopeptides) could stimulate secondary, systemic immune responses that could lead to a structural and functional repair and re-equilibration of the immune system. This effect could be termed a “modulation effect”, which we believe includes both “bridging” and “spreading” effects That is, we believe that a BLC according to the present invention could create a bridge to activate the innate and the acquired arms of the immune system. We expect that the activities of cytokines, mini-cytokines, nucleo-peptides and immunoactive peptides found in the material could have an effect in this regard.

In particular, if a subject were to ingest a dose of a BLC according to the present invention, we would expect to see one or more of the following clinical results:
1. an activation of the neutrophils in the production of immunoactive peptides of the innate immune system;
2. an activation of the cellular response of the acquired immune system; and
3. a restoration and normalization of the humoral response in the acquired immune system.
   We believe that these benefits of a BLC of the present invention would be significant since current medical research suggests that the restoration of immune function is a core factor in the recovery and maintenance of health.

Moreover, we believe that, in a healthy subject, an embodiment of an immunodynamic complex of the present invention could maintain, support and enhance the structure and function of the immune system.

Our current understanding of the “bridging” and “spreading” effects is described further below.

Immune system modulation is initiated via cytokine pathways and is a broad-spectrum effect that up-regulates and/or down-regulates as necessary to achieve optimum function of immune system surveillance, recognition, reaction and recovery. In an embodiment, the modulating effect of a BLC of the present invention is thought to be due to several particular classes of these small peptides. According to our understanding, some of the peptides act as a “bridge” between the innate and acquired arms of the immune system and may provide an opportunity to turn the immune system back on in immuno-compromised individuals.

We believe that there may be a feedback mechanism within the molecules in a BLC of an embodiment of the present invention (e.g., in particular defensins, granulysins, transfer factors and other nucleopeptides) that is capable of starting a system-wide immune cascade or “spreading effect.” Spreading effect is the term we use to describe a cytokine stimulation/activation of NK cells that we envision should happen when a BLC of the present invention is administered to a patient and operates to restore the damaged pathways. This spreading effect might be apparent if there were an increase in NK cell activity and NK cell numbers in a patient. An indication of cytokine involvement in a patient after the administration of a BLC in an embodiment of the present invention might be the increase in the amounts of alpha-TNF present in a patient's blood, since the amounts of alpha-TNF in a patient tend to increase when there is increased cellular communication by the T4 cells.

In addition, according to our understanding, a BLC in an embodiment of the present invention may cause macrophage activation and specific molecules to be generated by neutrophils. Providing this bridging action could be important in treating acute, severe, degenerative and some chronic diseases, where the immune system becomes so compromised as a result of the failure of key cytokine pathways, that CD4 cells become progressively compromised in their ability to generate an effective counter-attack. When this happens, we believe that an immune response has to be mounted circumventing the CD4 route. This can be done by administering, in an embodiment of the present invention, a BLC to increase the amount of macrophage, monocyte and natural killer cell activity until the cytokine pathways are restored.

### 2. A Systemic Look at the Immunology of a BLC

To start the cell-mediated immunity (CMI) response, the T-4 (helper) cells receive a signal from macrophages and/or dendritic cells that consume everything that they consider to be foreign to the host system. The T-4 cells can also be directly stimulated to take action by interacting with the antigens or through the activity of dendritic cells and some specialized B-cells. These helper cells are unarmed but once stimulated they send urgent biochemical signals (lymphokines) to special squadrons of T-cells called the lymphokine activated killer (LAK) cells and the natural killer cells (NK). When the alert signal comes in, LAK, NK and T4 cells start to multiply rapidly. We believe that transfer factors, as present, for example, in a BLC in an embodiment of the present invention, appear to offer an additional means of molecular communication for people with defective cell-mediated immunity.

Like all T-cells, natural killer cells (NK cells) are trained to recognize one specific enemy, such as a virally infected cell or a cancer cell. When called to action by the T-4 cells, the latent squadron replicates into an army with a single-minded task. The NK cells are lethal. When they attach to a target cell (e.g., such as a bacterium, virally infected cell or a cancer cell), a biochemical cascade is triggered that punctures the cell membrane, thus killing the target cell. Natural killer cells are a critical line of defense and often the body's only line of defense in cases of severe diseases and infections. Chronic stress, nutritional deficiencies and exposure to toxins can all suppress NK cells activity leaving one more vulnerable to infection or cancer.

The immune cascade is triggered when an antigen is recognized and the cells of the immune system connect one to another physically and by cytokine pathways to produce an end effect such as increased production of cytotoxic granules in the neutrophils, or an increase in natural killer cell function, an increase in LAK cells or an increased production of antibodies. This complex interplay of cells of the immune system with the connecting cytokine molecules is referred to as the immune cascade.

Depending upon the antigen, multiple pathways can be used to start the response. When multiple cytokine pathways are influenced simultaneously, the immune system tends to generate a more rapid and effective response. We believe that a BLC in an embodiment of the present invention contains immunoactive molecules that can either bypass areas of damage or activate cells through the actions of macrophages and dendritic cells that can start a cascade of events in the immune system that lead to an immune response. That is, we believe that a BLC in an embodiment of the present invention can stimulate a patient's immune system to reproduce or repair cellular membrane receptor sites, produce additional cytokines and restore cytokine pathways.

According to our understanding, cytokine pathway failure can be a precursor to and a symptom of many severe or chronic diseases, as reflected clinically upon studying a patient's full T & B cell subset panel. We expect that the effects of a BLC in an embodiment of the present invention might be demonstrated by a shift in a patient's T and B cell subset panel blood tests, as discussed in more detail in Examples 4. We also believe that the shift in a patient's T and B cell subset likely will be somewhat different from one patient to another because of all the different pathways and all the different cells that are involved and the variable pattern of damage. The multitude of immune cells in a patient, whether the patient is a person or animal, are thought to be connected to one another by a web of cytokine pathways and there are numerous effects that each cell can have depending upon what kind of cytokine stimulation and transfer factor information it receives. Furthermore, we believe that such shifts should happen over time, depending upon the pattern of damage to the person's immune system that can be monitored with T & B cell subset and NK function panels and other immune function tests.

In addition, we expect that a BLC in an embodiment of the present invention could cause immune markers to change, often unpredictably and dramatically, but generally should cause such markers to progress toward establishing a healthy immune system. This is an indication of what we term as “the modulation effect.” For example, this type of response might be noted in a patient with an immune counter-attack as in the case of a recovery from a B-cell virus, where the antibodies to that virus may initially go up temporarily before they come down. This up and down pattern of re-adjustment would be expected in blood tests of people who take a BLC in an embodiment of the present invention. Also, there may be a temporary decline in NK function before an increase is observed. The TNF marker may also go through an up and down movement as the immune system repairs itself. IGF-1 may increase significantly in a way that may initially create some concern but this again is an indication that the immune system is balancing itself. Consequently, during immune system support and reconstitution, the levels of various immune markers in a patient may change unpredictably in the early stages; however, we believe that this is simply reflective of the process of modulation.

Currently, conventional allopathic medical science does not know how to reconnect primary cytokine pathways. It is known how to block or bypass a pathway but not how to restore one. While all of the primary immune pathways are well understood in academic immunology, there is currently no area of research in modern medicine that deals with reestablishing a pathway. However, according to our understanding, a BLC in an embodiment of the present invention should be capable of supporting the restoration of primary cytokine pathways in a patient that play a critical role in the restoration and maintenance of health. Unfortunately, most people have some level of continual exposure to environmental and lifestyle stressors that stress the immune system and disrupt primary cytokine pathways. For many individuals, this may lead to a need for ongoing immune system support. We believe that an effective immune support is one that contains immunoactive molecules, such as found in a BLC in an embodiment of the present invention, that have been recognized to support cytokine pathways utilized by the body in building new cells. Poor nutrition, infection, toxins, trauma and stress (collectively referred to as the PITTS) have been shown to reduce the availability of these molecules, which would normally be available. There is a never ending battle between the PITTS and the immune system. As a result, optimal quantities of the immunoactive molecules may be lacking to support or maintain optimum function of the immune system and, we believe, may be furnished to a subject by administering an effective dose of a BLC in an embodiment of the present invention. Contemplated methods of using BLCs of the present invention are illustrated, for example, in Example 4.

### 3. Cytokine Pathways and a BLC

Monocytes become macrophages when monocytes and dendritic cells are activated. Moreover, macrophages and dendritic cells can activate both the innate immune system and the acquired immune system by inducing the immune cascade.

When macrophages and dendritic cells recognize an antigen, they produce interleukin-8 (IL-8). IL-8 activates the neutrophils to produce defensins, granulysins and other small immunoactive peptides. When macrophages are inactive and neutrophil activation is desired, IL-8 can be used to bypass that area of damage in the immune cascade. We believe that a BLC in an embodiment of the present invention may contain “IL-8-like” effects that can activate the neutrophils. Alternatively or additionally, we believe that such a BLC may contain inflammatory molecules, such as C5A that may be produced as a result of an irritation response in the udder to the antigen inoculation process. C5A is one of the complement factors that we understand activates macrophages and dendritic cells to produce IL-8 that, as noted before, are believed to activate the neutrophils. Our research to date discussed earlier regarding the protein/peptide content of a BLC in an embodiment of the present invention suggests that such a BLC contains molecules within the molecular weight range of IL-8.

We believe that other cytokines also exist in a BLC in an embodiment of the present invention. For example, looking at immune pathways that are activated, as evidenced by T and B cell activity, we also surmise that such a BLC may contain Interleukin-12 (IL-12) and Interleukin-4 (IL-4) or at least IL-12 and IL-4-like molecules. We believe that IL-4 may be an important interleukin for activating the pathways for the production of antibodies. IL-4 is thought to be in a pathway to which the immune system shifts when it needs to produce high levels of antibodies very quickly even though it may do so at the expense of shutting down other parts of the immune system.

We believe that a BLC in an embodiment of the present invention may also activate the adaptive immune system when administered to a human or animal. For example, as noted earlier, we believe that such a BLC may contain IL-12 or IL-12-like molecules, which activate immature T-cells, starting the immune cascade from the top. Such a BLC may also contain enough of the inflammatory agents and gamma-interferon to activate macrophages to produce IL-112 to start the cascade from that point.

The “bridge” from the adaptive or acquired immune system is thought to be activated through T-cells. We believe that a BLC in an embodiment of the present invention could create a “bridge” to activate the innate immune system and the acquired part of the immune system due to the activity of the cytokines and other communication molecules that may be induced by the BLC.

The following Examples are merely illustrative of the present invention and are not to be considered as limiting the invention, which is properly delineated in the following claims.

### EXAMPLES

### Example 1

### Preparation of Antigen/Cytokine Mixture

### Part 1—Antigen Preparation

The steps in this part were followed to prepare the antigen to be included in an antigen/cytokine mixture of the present invention. These steps make mention of the antigen as being a bacteria. However, we envision that any other type of antigen could also be used and then appropriately prepared.
1. A bacteria to be prepared into the antigen was suspended in ½ Normal Saline
2. A swab was used to coat the bacteria evenly onto a 5% SRBC plate.
3. The plate was incubated for 24 hours to establish a thick growth in log phase without using up all of the available nutrients.
4. A swab was used to harvest the bacteria and the bacteria was then suspended in deionized water to a concentration of 28% light transmission. We found that one petri dish could prepare about 20 cc of the suspension.
5. 0.01 cc (10 ul) of HCl per 20 ml of bacteria was added to the bacterial suspension. (NOTE: Our research has shown that our technique can produce sterile results at concentrations of 10 ul HCl:40 cc of bacterial suspension. However, conservatively, a working concentration of 10 ul: 20 cc was used.)
6. The bacterial solution was incubated in a CO₂ incubator at 37 C for 4 hours.
7. The bacterial solution was then placed in a super freezer (−80 C) for 48 hrs.
8. Later, the solution was thawed, tested for sterility and quality (see below) and used as needed.

The procedure described above for preparing an antigen, specifically a bacteria for use in the antigen/cytokine mixture, is beneficial for a number of reasons. First, the final solution is slightly acidic, which is immunologically advantageous when mixed with cytokines. Second, incubating the bacteria at 37 C stimulates them to grow and divide further. This makes them more permeable for the absorption of nutrients. Third, putting them in deionized water, which is an extremely hypotonic fluid, will cause them to swell. Then putting them in a super freezer will cause ice crystals to form in and through them, thereby causing them to lyse and explode into protein fragments. Our microscopic evaluation of the antigen solution as prepared above demonstrated cell fragments with only very rare “intact cells” seen. Protein fragments tend to be easier to effectively use in an inoculation gun because they are smaller and can penetrate through intact skin better as compared to whole cells.

### Part 2—Sterility Testing of Antigen Prior to Its Introduction Into the Animal

Following its preparation, the antigen was tested to ensure that it was sterile. The sterility testing described in this part is one of several methods that we have developed to ensure that a BLC of the present invention is of a high quality.
1. A loop was used to evenly coat a petri dish with the thawed solution from above. The petri dish was then incubated in a CO₂ incubator at 37 C for 48 hrs.
2. As a second test, a standard solution of Todd Hewitt media (30 gms in 1 litre) was prepared.
3. 100 cc of the Todd Hewitt media was placed in a sterile flask and 5 cc of the bacteria solution as prepared above was added.
4. The flask from step 3 was incubated in a CO₂ incubator at 37 C for 48 hrs.
5. If the incubated solution was still clear, a swab was used to evenly coat a petri dish with the Todd Hewitt solution from above and this petri dish was then incubated in a CO₂ incubator at 37 C for 48 hrs.
6. If the petri dishes were still clear after 48 hrs, then the prepared bacterial antigens were considered sterile.

### Part 3—Testing the Quality of an Antigen

This assay was designed to determine whether an antigen is immunologically recognizable once it has been prepared. Knowing that the antigen is immunologically recognizable provides some assurance that the inoculation will be effective if the gun-inoculation is then properly administered. This is a very useful quality assurance procedure that we have developed. We envision using this procedure as part of a method of preparing an immunodynamic complex, including a BLC in an embodiment of the present invention. We also believe that this test could be very valuable in other applications, such as in the vaccine industry generally to test the quality of antigens that are used to prepare vaccines.

1. An antigen to be tested was prepared, for example as described in Parts 1 and 2. Thereafter, a standard cell culture medium was prepared as described further below.
2. The following materials were assembled:

Reagents:

RPMI 1640 complete culture media (Cellgro.com).

Fetal bovine serum (FBS)

Pennicillin-Streptomycin Stock (5000 IU and 5000 ug/mL)

L-glutamine stock (200 mM in DI H₂O)

Materials/Instruments:

Sterile pipets/pipettor tips

Sterile 25 mL culture flasks

Biosafety hood

CO₂ incubator set at 37° C.

Centrifuge

15 mL conical tubes

Sysmex SF-3000

3. A sufficient amount of RPMI 1640 was prepared and was supplemented with 10% FBS, 2 mM of L-glutamine, and 100 ug/mL of penicillin-streptomycin. This was accomplished by adding 5 ml of FBS, 500 uL of L-glutamine stock ([200 mM] in DI) and 100 uL of antibiotic stock (Penicillin/Streptomycin) to fill to 50 ml with liquid RPMI 1640 medium. The complete medium was stored refrigerated for no longer than three weeks.
4. The bio-safety hood was sterilized (with UV light) and was turned on for a minimum of 15 minutes before use.
5. All necessary supplies were placed under the hood and only sterile supplies were used. Sterile technique was used to transfer all liquids and cell suspensions.
6. 8 mL of prepared medium was pipetted into each of two flasks to be inoculated.
7. The culture medium was examined visually for any signs of contamination. If there were any signs of bacterial or fungal contamination, the culture medium was not used.
8. (6) 10 cc amounts of blood were drawn from a human volunteer (although bovine, or other mammalian, blood also could have been used) in ACD (yellow top) tubes.
9. The tubes were centrifuge at 1800 RCF for 5 minutes.
10. Using a pipette, the “buffy coat” was drawn off and placed in a sterile test tube.
11. The white blood cell (WBC) count in the samples was verified with a (Coulter) cell counter so that the final concentration of cells in the culture was between about 0.1−1.0×10⁶.
13. 1 cc of the cells was drawn up and added to each flask with 8 cc of cell culture medium.
14. 1 cc of antigen was added (pH adjusted to the pH of the cell culture medium) (to give a concentration of 10%) to one of the cell cultures. Both cell cultures were then incubated in a CO₂ incubator at 37 C for 5 hours.
15. The cells were then stained (labeled) with CD 45 to differentiate the WBCs from red blood cells (RBCs).
16. The cells were also stained (labeled) with CD 25, an activation marker.
17. The two cell culture samples were compared using flow cytometry for relative levels of activation response.

By following these steps, an antigen to be inoculated could be identified as immunologically recognizable and thus appropriate for gun inoculation. For example, the staining for CD 45 separated RBCs from WBCs, which identified the population of cells to monitor using the flow cytometer. Thereafter, the staining for CD 25 revealed the shift in grid position of the WBC. That is, on the flow cytometer graph, there was evidence of the leucocytes migrating above the horizontal line of the graph. This indicated that the WBCs were positive for the CD 25 marker, meaning that they were responsive to the antigen, which, in turn, demonstrated a cellular activation in response to antigen recognition. The control sample did not show this migration, indicating that there was no antigenic response. Although in this experiment we used CD 25 as an activation marker, it should be recognized that other activation markers can also be used, such as CD 69, as illustrated in Part 3 of Example 5 below.

### Part 4—Calculating the Amounts of Cytokine Required for Antigen/Cytokine Mixture

In addition to preparing the antigen, it is also necessary to prepare the cytokine mixture that will be used. We used the following cytokines in preparing the cytokine mixture:
Recombinant Bovine TNF Alpha (Alpha-TNF) (e.g., available from Endogen or from Pierce Biotech)
Recombinant Bovine IFN Gamma (G-INF) (e.g., available from Serotec)
Interleukin-8 human (IL-8) (e.g., available from Sigma)
Granulocyte-Macrophage Colony-Stimulating Factor (GM-CSF) (e.g., available from Sigma).

The potency of the cytokines were measured as an Effective Concentration 50 (EC 50), namely, the concentration required for 50% inhibition or activation of the cells involved. In view of this, the amount of cytokine that we needed for the antigen/cytokine mixture of this Example was determined according to the following rationale:
1. Since the EC 50 is the amount needed to activate ½ of the cells involved, this concentration was doubled to aim for 100% activation. (Since most cytokines have a bi-phasic response curve, giving too much could have had the opposite effect of what we desired.)
2. Since some material will be lost on the wall of the vial and in the wall of the udder, twice the 100% activation amount was determined to be needed, which was thus four times the EC 50.
3. Since material likely would be lost as it transversed the udder wall, it was determined that the amount from step 2 should be doubled to attain a grand total of 8 times the EC 50.
4. The concentration of each cytokine to be used was determined based generally on the following calculations.

| Alpha - TNF | G - INF | IL-8 | GM-CSF |
|---|---|---|---|
| 20,000 ng/ml | 300 ng/ml | 10,000 ng/ml | 5,000 ng/ml amt rec'd |
| EC 50 = | EC 50 = | EC 50 = | EC 50 = |
| 10 ng/ml | 10 ng/ml | 222 ng/ml | 1 ng/ml |

Thus using 2×EC 50 for approx 100% activation:

| Alpha - TNF | G - INF | IL-8 | GM-CSF |
|---|---|---|---|
| 20 ng/ml | 20 ng/ml | 444 ng/ml | 2 ng/ml |

To compensate for at least a loss of 50% of the material as it goes through the tissues and another 50% loss of material that sticks to the sides of the vial we thus obtained:

| Alpha - TNF | G - INF | IL-8 | GM-CSF |
|---|---|---|---|
| 80 ng/ml | 80 ng/ml | 1776 ng/ml | 8 ng/ml |

5. In view these desired concentrations and the fact that 0.1 ml (i.e. 0.1 cc) of each cytokine was desired, it was determined that the following volume of liquid should be added to reconstitute the commercially available cytokine.

### Alpha-TNF

if 0.1 ml is taken of each cytokine in the above concentrations, so to reconstitute:

$$\frac{20 \text{,} 000}{X} : \frac{80}{0.1}$$

X=25 ml of additional volume was needed

### Gamma-Interferon

$$\frac{300}{X} : \frac{80}{0.1}$$

X=0.375 ml of additional volume was needed

### Interleukin-8

$$\frac{10 \text{,} 000}{X} : \frac{1776}{0.1}$$

X=0.56 ml of additional volume was needed

### Granulocyte Monocyte—Colony Stimulating Factor

$$\frac{5 \text{,} 000}{X} : \frac{8}{0.1}$$

X=62.5 ml of additional volume was needed

### Part 5—Preparation of Antigen/Cytokine Mixture

Having determined the amount of cytokine required in Part 4, the antigen/cytokine mixture was prepared as described further below.

1. 100 cc of “Fisherbrand Blood Bank Saline cat #23-062-125” and add 1 gm of Albumin bovine serum (Bovine albumin or BSA) e.g., available from Sigma) was obtained.
Note: An appropriate amount of protein was added as a carrier molecule to keep the cytokines in solution and to make sure that they did not just adhere to the wall of the vial. Bovine serum albumin was found to be a good carrier molecule. For example, it was found that, if the solution had too much protein and was thus too viscous, it would be too thick and would not penetrate deep enough into and through the udder to be effective. On the other hand, if there was too little protein, then an unacceptable amount of cytokines might be lost, for example, due to wall adherence, to be effective.
   2. The saline/BSA was the aliquoted as follows in order to appropriately reconstitute the commercially available cytokine as described in Part 4:
A) 25 ml of the bovine serum albumin for the Alpha-TNF in a sterile cell culture vial;
B) 0.38 ml of the bovine serum albumin for the G-INF in a test tube;
C) 0.6 ml of the bovine serum albumin for the IL-8 in a test tube; and
D) 62.5 ml of the bovine serum albumin for the GM-CSF in a sterile cell culture vial
   3. Using separate 0.5 cc syringes, at least 1 of the syringe of the buffered saline/BSA was drawn up from each of the above test tubes or sterile cell culture vials to be used in step 5.
   4. In each of the above test tubes or sterile cell culture vials, the lyophilized powder/pellet of the appropriate cytokine was added.
   5. Using the appropriate syringe, the drawn up buffered saline/BSA from step 3 was used to “wash out” what powder remained.
   6. Once all of the powder was reconstituted, the test tubes and sterile cell culture vials were then placed in a Vortex Mixer, and set on about 7, for 15 seconds.
   7. Using a 5 cc syringe, 2 cc of the antigen were drawn up and added it to the glass injection vial.
   8. Then using the 0.5 cc syringe, 0.4 cc of each cytokine was drawn up individually from the mixing vial or test tube and placed in the glass injection vial.
   9. The glass injection vial then had sufficient antigen/cytokine mixture for four inoculations, where:

The final quantities of antigen/cytokine mixture for each inoculation was:
0.5 cc of antigen
0.1 cc of each of the 4 cytokines

For a total volume of 0.9 cc per inoculation.

### Example 2

### Manufacturing a BLC

Once the antigen/cytokine mixture was prepared as discussed in Example 1, the following steps were taken to prepare a BLC.

1. The antigen/cytokine mix was placed into an inoculation gun with a setting sufficient to penetrate the udder wall. The inoculation gun pressure settings were set at 400 psi on the 2 front quarters and 650 psi on the 2 rear quarters. Moreover, the inoculation gun volume settings were set at 0.9 cc on all four quarters, in order to deliver the 4 doses of the antigen/cytokine mixture prepared as described in Example 1.

2. The antigen/cytokine mix was then injected into the udder, for example, into each quarter of a lactating cow's or other ungulate's udder. If desired, the subject cows were placed on a specified regimen of dietary supplements prior to the injection. The inoculation gun was placed at a point on the side of each quarter where there was no vein and pressed firmly against the skin to administer the cytokine/antigen mixture directly into the udder through the wall of the udder. The inoculation gun was placed 2-3 inches above the base of the teat, and not over a vein, making sure that the nose of the gun was flat against the skin and under the udder hair.

3. Starting two days after the antigen/cytokine gun inoculation, the “milk product” was collected and the fat was removed by either a centrifugal cream separator or by letting the milk stand undisturbed until the cream rose, following which the milk was decanted off the bottom of the tank.

4. Although not necessary, the milk could then be frozen for a sufficient time to facilitate cell lysis, which typically was not less than three days.

5. When ready for processing, the milk was gradually thawed at room temperature.

6. The milk was then pasteurized using: batch pasteurization at 145 F for a minimum of 30 minutes; flow through pasteurization at 160 F for a minimum of 15 seconds; or any other acceptable pasteurization method at suitable temperatures and times.

7. The milk was then decaseinated, i.e. the curds were removed. Decaseination was accomplished by lowering the pH using hydrochloric acid or an organic acid, e.g. proprionic acid, or by more traditional methods used in cheese making, such as the addition of rennet. This step occurred at a temperature of 110 F.

8. The milk solids were then removed so that only whey remained. A strainer or scoop with holes removed the bulk of the solids. A small strainer or a filtering cloth removed the smaller solids.

9. The whey was then filtered with a hollow fiber micro filter or using ultrafiltration so as to remove the larger proteins, globulins, large molecules, residual fat, microbes and contaminates, thereby leaving a filtrate containing the desired molecules of 100 kD or less.

10. Microbiology testing was then conducted for salmonella, E. coli, Staph aureus and aerobic plate count, as described further in Example 3.

11. For production, the extract was allowed to thaw to about 50% and was then placed in a jacketed stainless steel vat that the could be used to heat or cool the vat contents, as desired.

12. A second pasteurization was performed just prior to the drying step.

13. The extract was placed in clean, sanitized plastic pails or other appropriate containers and was placed in a freezer for storage until further processing. The product that was designated for spray drying or freeze drying was concentrated to a higher solids content using a reverse osmosis dairy filter. This step removed water from the whey, concentrating the solids from approximately 6% to 17-20%.

### Example 3

### Microbiology Testing Using a BLC

Microbiology testing was undertaken using a BLC of the present invention as prepared in Example 2, or portions thereof, as described in this Example. Part 1 of this Example shows how the BLC was prepared in order to be used for the microbiology testing delineated in Parts 2 through 7. However, since Parts 4 to 7 of this Example were conducted specifically using defensins, granulysins, lactoferrin or transfer factors found in the BLC, the processed BLC prepared in Part 1 was separated by gel electrophoresis into its various protein/peptide components prior to such testing being conducted.

### Part 1—Preparation of Lacteal Secretions for Laboratory Testing

Prior to conducting any laboratory testing, the BLC obtained as described in Example 2, was processed as described here. All lacteal secretions resulting from traditional infusion (i.e. infusing an antigen through the teat canal) that were used in the antimicrobial testing described below were processed in the same manner. In addition, such lacteal secretions obtained from traditional infusion were previously processed to remove the fats, as well as to remove the large molecular weight proteins over 100 kD.

1. Initially, an acetate buffer 3.15 N, having a pH=4.3, was prepared by adding 80 ml of 3.15 N acetic acid solution (i.e., 18 ml glacial acetic acid to 100 ml deionized H₂O, pH=2.26) to 20 ml of 3.1 N sodium acetate solution (i.e., 40.8 g sodium acetate trihydrate to 100 ml H₂O).
NOTE: An organic acid buffer system was used (instead of HCl) because it is gentler on the small proteins whose activity are being tested in the laboratory tests described below. It is less likely that an organic acid buffer system will denature such proteins.

2. If previously frozen, the lacteal secretion sample was allowed to thaw at room temperature before acid treatment. After thawing, the sample was mixed well by shaking vigorously, and then approx. 80 mls were transferred to a sterile specimen container containing 10 ml of acetate buffer. A corresponding label was attached to this container.

3. The acidified lacteal secretion sample was mixed by shaking gently, and was then allowed to rest at room temperature for 15 to 30 min. to complete the coagulation.

4. A 40 ml aliquot of the acidified lacteal secretion sample was then removed into a disposable 50 ml conical polypropylene centrifuge tube and was centrifuged at 1800×g for 5 min.

5. After centrifugation, about 20 ml of the supernatant was decanted into a filter apparatus containing a 0.2 μm membrane filter. (The filtering apparatus used was made by Nalge Corp., containing a pre-assembled Nalgene (Polystyrene), 115 ml sterile vacuum filter unit with a 0.2 u (Micron) filter in place, part # 124-0020) The supernatant was then filtered with suction until 15 ml of filtrate was obtained.

6. The filtrate was transferred to a sterile labeled disposable screw-capped 15 ml centrifuge tube. The pH of the filtrate was verified using a pH meter calibrated between pH 4.0 and 7.0. Typically, a pH of 4.5±0.3 was measured for the filtrate.

7. The filtrate was then stored at 4° C., awaiting its use in various protocols, including gel electrophoresis, antimicrobial testing and mass spectroscopy analysis.

### Part 2—Antimicrobial Activity of a BLC

After processing a BLC sample according to Part 1 of this example, the antimicrobial activity of the BLC sample was tested as against a traditional lacteal secretion as described below. These results show that a BLC prepared by the method of Example 2 is more antimicrobially effective than a lacteal secretion sample obtained from a traditionally infused cow. It is important to note that this procedure was designed to test for an antimicrobial effect specific to a test bacteria which is the same as the bacteria used as the antigen in the antigen/cytokine mixture prepared in Example 1 (or the antigen used in the production of a lacteal secretion from a traditionally infused cow).

The following materials were used:
½ Normal Saline (NS)
TSA ll 5% SRB Agar plate (BBL prepared media), 10-30 plates depending an the testing to be done.
Processed lacteal secretions as prepared in Part 1: (1) a BLC sample and (2) traditionally infused sample
Active (in log phase) culture of a pathogenic strain of group A Beta hemolytic Streptococcus phenotype Strep. Pyogenes, ATCC # 19615.
Small glass test tube for diluting the bacteria
1 ul culture loops
Vitek Colorimeter is needed to approximately standardize the concentration of bacteria to be tested (i.e., the Colorimeter was zeroed using ½ Normal Saline to align both ends of the instrument's scale)

The following method was used:

1. The bacteria to be tested were previously plated and grown on a standard TSA ll 5% SRB Agar plate (BBL prepared media) and incubated in a CO₂ Napco model 5400 incubator at 37 C and 5.0% CO2. In this case, an active (in log phase) culture of a pathogenic strain of group A Beta hemolytic Streptococcus phenotype Strep. Pyogenes, ATCC # 19615 was used.

2. On the testing day, enough bacteria was added to a test tube of 1 NS (0.45% Baxter), with a sterile cotton swab, to decrease the solution to 95% transmissible on a Vitek Colorimeter (after being zeroed with ½ NS). When the bacteria were added, the swab was twirled and the solution was allowed to settle before being measured. This is further referred to as the stock solution (SS).

3. After alcohol swabbing the top of each test sample bottle, 5 ul or 10 ul of a test sample (i.e., (1) a BLC sample and (2) a traditionally infused sample) was taken and mixed with increasing volumes (in a 1.5 cc capped micro centrifuge tube) of the SS to make the dilutions as follows:
10 ul Test Sample: 100 ul SS=1:11
5 ul Test Sample: 250 ul SS=1:51
5 ul Test Sample: 450 ul SS=1:91
4. The solutions were immediately plated (at time 0) and then put into the incubator until needed 15 minutes later. The incubation time was then tracked in 15 minute increments in order to be able to reproducibly track any response.

5. After the time of incubation, using a second incubated tube rack to take the needed tube out of the incubator so as to maintain as constant a temperature as is possible and utilizing a quantifying loop (1:1000, 1/1000 of a ml, 1 ul Nunc brand radiation sterilized, disposable), a sample was taken (being careful to touch the loop to the inside of the tube to draw off any excess) of the mixture and plated on ½ of a SRB agar plate. The other ½ was plated with 1 ul of the SS or a second test product (e.g.) and further incubated at 37 C.

6. After 24 hours, the dishes were removed from the incubator and “read.”

7. A control plate was run after all the test plates were done by taking a simple loop of the bacteria from the stock solution in the test tube and plating out to ensure that the bacteria did not die during the experiment.

The results provided in Table 1 below sets forth the rate at which a BLC and a traditionally infused lacteal secretion were able to inhibit the bacterial growth. As a control, an uninfused sample (i.e., normal milk) was also tested and showed no growth inhibition on preliminary testing. In the table, T₀ to T₉₀ reflects measurements taken at zero minutes through 90 minutes. Moreover, the identifiers “91”, “151”, and “11” reflect the dilutions of the sample that were used in the plates being tested. Also, the identifiers “1” and “2” reflect results for the two different samples that were tested. That is, sample 1 is a traditional lacteal secretion sample, whereas sample 2 is a BLC sample according to the present invention.

TABLE 1

| | | T₀ | T₁₅ | T₃₀ | T₄₅ | T₆₀ | T₇₅ | T₉₀ |
|---|---|---|---|---|---|---|---|---|
| 91 | 1 | >200K | >200K | >200K | >200K | 172K | 97K | 30K |
| | | CFU/mL | CFU/mL | CFU/mL | CFU/mL | CFU/mL | CFU/mL | CFU/mL |
| | 2 | >200K | >200K | 88K | 27K | 3K | 0 | 0 |
| | | CFU/mL | CFU/mL | CFU/mL | CFU/mL | CFU/mL | CFU/mL | CFU/mL |
| 51 | 1 | >200K | >200K | >200K | 182K | 64K | 5K | 0 |
| | | CFU/mL | CFU/mL | CFU/mL | CFU/mL | CFU/mL | CFU/mL | CFU/mL |
| | 2 | >200K | 124K | 39K | 4K | 0 | 0 | 0 |
| | | CFU/mL | CFU/mL | CFU/mL | CFU/mL | CFU/mL | CFU/mL | CFU/mL |
| 11 | 1 | >200K | 191K | 34K | 0 | 0 | 0 | 0 |
| | | CFU/mL | CFU/mL | CFU/mL | CFU/mL | CFU/mL | CFU/mL | CFU/mL |
| | 2 | +/−200K | 18 | 0 | 0 | 0 | 0 | 0 |
| | | CFU/mL | CFU/mL | CFU/mL | CFU/mL | CFU/mL | CFU/mL | CFU/mL |

Note:
the controls grew out positive >200K.
CFU/ml = colony forming units (in thousands) per 1 cc volume

The BLC sample was substantially more antimicrobial than the traditionally infused sample. The strongly antimicrobial nature of the BLC sample was particularly apparent from the rate of inhibition in the 1:91 diluted samples. After one hour, the traditionally infused sample had only reduced the number of CFU/ml such that 172,000 were left remaining; however, after half an hour, only 88,000 CFU/ml were left in the BLC sample. Consequently, the rate of inhibition, and the amount of inhibition in a certain time period reveal that the BLC sample could easily be considered to be at least 2 to 2.5 times more antimicrobial than the traditionally infused sample.

### Part 3—Laboratory Testing of a BLC Against Pathogenic and Nonpathogenic Bacteria

The procedure described in Part 2 was also used here to conduct antimicrobial testing that demonstrates that a BLC is capable of inhibiting the growth of pathogenic strains of bacteria but not the growth of nonpathogenic strains of bacteria. This is very useful since it demonstrates that a BLC is capable of antimicrobial activity with great specificity. We expect that such a characteristic would be highly advantageous in a clinical setting, since patients being administered a BLC as described in further detail in Examples 4-7 should not get a sterile gut as a result of treatment, which can lead to stomach upset.

In this experiment, a BLC sample prepared according to the present invention was used as against both a pathogenic and a nonpathogenic strain of bacteria. Table 2 set forth below provides the results of this testing. In Table 2,

1=Sample 1—the bacteria was non pathogenic Alpha Strep

2=Sample 2—the bacteria was Beta hemolytic, pathogenic Strep

The results were as follows:

Sample 1 did not show any growth inhibition

Sample 2 showed a typical bactericidal effect as seen in other BLC samples

Also, the same designations used in Table 1 are also relevant in Table 2, namely:

T=Minutes

10 ul Test Sample: 100 ul SS=1:11 (denoted in Table 2 as “11”)

5 ul Test Sample: 250 ul SS=1:51 (denoted in Table 2 as “51”)

5 ul Test Sample: 450 ul SS=1:91 (denoted in Table 2 as “91”)

CFU/ml=colony forming units (in thousands) per 1 cc volume

TABLE 2

| | | T₀ | T₁₅ | T₃₀ | T₄₅ | T₆₀ | T₇₅ | T₉₀ |
|---|---|---|---|---|---|---|---|---|
| 91 | 1 | >200K | >200K | >200K | >200K | >200K | >200K | >200K |
| | | CFU/mL | CFU/mL | CFU/mL | CFU/mL | CFU/mL | CFU/mL | CFU/mL |
| | 2 | >200K | >200K | >91K | 38K | 7K | 0 | 0 |
| | | CFU/mL | CFU/mL | CFU/mL | CFU/mL | CFU/mL | CFU/mL | CFU/mL |
| 51 | 1 | >200K | >200K | >200K | >200K | >200K | >200K | >200K |
| | | CFU/mL | CFU/mL | CFU/mL | CFU/mL | CFU/mL | CFU/mL | CFU/mL |
| | 2 | >200K | >132K | 25K | 9K | 0K | 0 | 0 |
| | | CFU/mL | CFU/mL | CFU/mL | CFU/mL | CFU/mL | CFU/mL | CFU/mL |
| 11 | 1 | >200K | >200K | >200K | >200K | >200K | >200K | >200K |
| | | CFU/mL | CFU/mL | CFU/mL | CFU/mL | CFU/mL | CFU/mL | CFU/mL |
| | 2 | +/−200K | 12K | 0 | 0 | 0 | 0 | 0 |
| | | CFU/mL | CFU/mL | CFU/mL | CFU/mL | CFU/mL | CFU/mL | CFU/mL |

Part 4—Antimicrobial Activity of Defensins Derived from a BLC

This experiment demonstrates that the defensins produced in a BLC of the present invention have specific antimicrobial activity. In order to conduct this experiment, a BLC sample was first processed in accordance with the procedure described in Part 1 of this Example, and the resulting processed sample was then separated by gel electrophoresis on a tris-glycine gel, which preserve the native protein's function. Thereafter, reverse electrophoresis was conducted on the defensin band, which is band # 12 in FIG. 1, to remove and purify this band. Although transfer factors remained in this band, it is our understanding of common immunological knowledge that transfer factors do not exhibit any antimicrobial activity. The defensins were then suspended in a saline solution. Antimicrobial testing using the method set forth below was then undertaken.

1. The bacteria (i.e., Streptococcus pyogenes) to be tested were previously plated and grown on a standard TSA ll 5% SRB Agar plate (BBL prepared media) and incubated in a CO₂ Napco model 5400 incubator at 37 C and 5.0% CO₂. On the testing day, enough bacteria were added to a test tube of ½ NS (0.45% Baxter), with a sterile cotton swab, to decrease the solution to 95% transmissible on a Vitek Colorimeter (after being zeroed with ½ NS). When the bacteria were added the swab was twirled and the solution was allowed to settle before being measured. This is further referred to as the stock solution (SS).
2. 50 ul of the SS was mixed with 50 ul of the sample to be tested to thus give a dilution of 1:2, other dilutions were similarly prepared. This “large” volume was used for ease of manipulation.
3. This solution was put back into the incubator for timed intervals.
4. After the time of incubation, utilizing a quantifying loop (1:1000, 1/1000 of a ml, 1 ul Nunc brand radiation sterilized, disposable) was taken (being careful to touch the loop to the inside of the tube to draw off any excess) of the mixture and plated on ½ of a SRB agar plate, the other ½ was plated with 1 ul of the SS and further incubated at 37 C.
5. After 24 hours the dish was removed from the incubator and “read”.

The results were as follows:

The uninfused sample (i.e., control) did not show any bactericidal activity.

The traditionally infused sample was bactericidal.

The BLC sample was highly bactericidal.

Note: The control samples which was uninfused material (i.e., normal milk) did not show any bactericidal activity and grew out positive>200K.

The results of this antimicrobial testing are provided in Table 3. The following abbreviations are used in Table 3:

1=Sample 1—From traditionally infused cow.

2=Sample 2—From gun-inoculated cow (i.e., a BLC)

T=Minutes

10 ul Test Sample: 100 ul SS=1:11 (denoted in Table 3 as “11”)

5 ul Test Sample: 250 ul SS=1:51 (denoted in Table 3 as “51”)

5 ul Test Sample: 450 ul SS=1:91 (denoted in Table 3 as “91”)

CFU/ml=colony forming units (in thousands) per 1 cc volume

TABLE 3

| | | T₀ | T₁₅ | T₃₀ | T₄₅ | T₆₀ | T₇₅ | T₉₀ |
|---|---|---|---|---|---|---|---|---|
| 91 | 1 | >200K | >200K | >200K | >200K | 172K | 97K | 30K |
| | | CFU/mL | CFU/mL | CFU/mL | CFU/mL | CFU/mL | CFU/mL | CFU/mL |
| | 2 | >200K | >200K | 88K | 27K | 3K | 0 | 0 |
| | | CFU/mL | CFU/mL | CFU/mL | CFU/mL | CFU/mL | CFU/mL | CFU/mL |
| 51 | 1 | >200K | >200K | >200K | 182K | 64K | 5K | 0 |
| | | CFU/mL | CFU/mL | CFU/mL | CFU/mL | CFU/mL | CFU/mL | CFU/mL |
| | 2 | >200K | 124K | 39K | 4K | 0 | 0 | 0 |
| | | CFU/mL | CFU/mL | CFU/mL | CFU/mL | CFU/mL | CFU/mL | CFU/mL |
| 11 | 1 | >200K | 191K | 34K | 0 | 0 | 0 | 0 |
| | | CFU/mL | CFU/mL | CFU/mL | CFU/mL | CFU/mL | CFU/mL | CFU/mL |
| | 2 | +/−200K | 18 | 0 | 0 | 0 | 0 | 0 |
| | | CFU/mL | CFU/mL | CFU/mL | CFU/mL | CFU/mL | CFU/mL | CFU/mL |

Note:
the control samples which was un-infused material grew out positive >200K.

Part 5—Antimicrobial Activity of Granulysins Derived from a BLC

This experiment demonstrates that the granulysins produced in a BLC according to the present invention have antimicrobial activity. In order to conduct this experiment, a BLC sample was first processed in accordance with the procedure described in Part 1 of this Example, and the resulting processed sample was then separated by gel electrophoresis on a tris-glycine gel, which preserves the protein's native function. Thereafter, reverse electrophoresis was conducted on the granulysin band, which is band # 11 in FIG. 1, to remove and purify the granulysins from this band. The purified granulysins were then suspended in a saline solution. Antimicrobial testing using essentially the same method as set forth in Part 4 of this Example was then undertaken.

The results were as follows:

The uninfused sample (i.e., control) did not show any bactericidal activity. The traditionally infused sample was bactericidal.

The gun-inoculated sample (i.e., a BLC) was highly bactericidal.

Note: the control samples which was un-infused material did not show any bactericidal activity and grew out positive>200K CFU/mL.

The results of this antimicrobial testing are provided in Table 4. The following abbreviations are used in Table 4:

1=Sample 1—From traditionally infused cow.

2=Sample 2—From gun-inoculated cow (i.e., a BLC)

T=Minutes

10 ul Test Sample: 100 ul SS=1:11 (denoted in Table 4 as “11”)

5 ul Test Sample: 250 ul SS=1:51 (denoted in Table 4 as “51”)

5 ul Test Sample: 450 ul SS=1:91 (denoted in Table 4 as “91”)

CFU/ml=colony forming units (in thousands) per 1 cc volume

TABLE 4

| | | T₀ | T₁₅ | T₃₀ | T₄₅ | T₆₀ | T₇₅ | T₉₀ |
|---|---|---|---|---|---|---|---|---|
| 91 | 1 | >200K | >200K | >200K | >200K | >200K | >200K | 173K |
| | | CFU/mL | CFU/mL | CFU/mL | CFU/mL | CFU/mL | CFU/mL | CFU/mL |
| | 2 | >200K | >200K | >200K | >200K | >200K | 157K | 85K |
| | | CFU/mL | CFU/mL | CFU/mL | CFU/mL | CFU/mL | CFU/mL | CFU/mL |
| 51 | 1 | >200K | >200K | >200K | >200K | 163K | 5K | 0 |
| | | CFU/mL | CFU/mL | CFU/mL | CFU/mL | CFU/mL | CFU/mL | CFU/mL |
| | 2 | >200K | >200K | >200K | 188K | 69K | 0 | 0 |
| | | CFU/mL | CFU/mL | CFU/mL | CFU/mL | CFU/mL | CFU/mL | CFU/mL |
| 11 | 1 | >200K | >200K | 134K | 31 | 4 | 0 | 0 |
| | | CFU/mL | CFU/mL | CFU/mL | CFU/mL | CFU/mL | CFU/mL | CFU/mL |
| | 2 | >200K | +/−200K | 49K | 0 | 0 | 0 | 0 |
| | | CFU/mL | CFU/mL | CFU/mL | CFU/mL | CFU/mL | CFU/mL | CFU/mL |

Part 6—Antimicrobial Activity of Lactoferrin Derived from a BLC

This experiment demonstrates that the lactoferrin produced in a BLC according to the present invention has antimicrobial activity. In order to conduct this experiment, a BLC sample was first processed in accordance with the procedure described in Part 1 of this Example, and the resulting processed sample was then separated by gel electrophoresis on a tris-glycine gel, which preserves the protein's native function. Thereafter, reverse electrophoresis was conducted on the lactoferrin band, which is band # 2 in FIG. 1, to remove and purify the lactoferrin from this band. The purified lactoferrin was then suspended in a saline solution. Antimicrobial testing using essentially the same method as set forth in Part 4 of this Example was then undertaken.

The results were as follows:

The uninfused sample (i.e., control) did not show any bactericidal activity.

The traditionally infused sample was bactericidal.

The gun-inoculated sample (i.e., a BLC) also was bactericidal.

Note: the control samples which was un-infused material did not show any bactericidal activity and grew out positive>200K CFU/mL

The results of this antimicrobial testing are provided in Table 5. The following abbreviations are used in Table 5:

1=Sample 1—From traditionally infused cow.

2=Sample 2—From gun-inoculated cow.

T=Minutes

10 ul Test Sample: 100 ul SS=1:11 (denoted in Table 5 as “11”)

5 ul Test Sample: 250 ul SS=1:51 (denoted in Table 5 as “51”)

5 ul Test Sample: 450 ul SS=1:91 (denoted in Table 5 as “91”)

CFU/ml=colony forming units (in thousands) per 1 cc volume

TABLE 5

| | | T₀ | T₁₅ | T₃₀ | T₄₅ | T₆₀ | T₇₅ | T₉₀ |
|---|---|---|---|---|---|---|---|---|
| 91 | 1 | >200K | >200K | >200K | >200K | >200K | >200K | >200K |
| | | CFU/mL | CFU/mL | CFU/mL | CFU/mL | CFU/mL | CFU/mL | CFU/mL |
| | 2 | >200K | >200K | >200K | >200K | >200K | >200K | >200K |
| | | CFU/mL | CFU/mL | CFU/mL | CFU/mL | CFU/mL | CFU/mL | CFU/mL |
| 51 | 1 | >200K | >200K | >200K | >200K | >200K | 177K | 85K |
| | | CFU/mL | CFU/mL | CFU/mL | CFU/mL | CFU/mL | CFU/mL | CFU/mL |
| | 2 | >200K | >200K | >200K | >200K | >200K | 194K | 90K |
| | | CFU/mL | CFU/mL | CFU/mL | CFU/mL | CFU/mL | CFU/mL | CFU/mL |
| 11 | 1 | >200K | >200K | >200K | >200K | 163K | 72K | 0 |
| | | CFU/mL | CFU/mL | CFU/mL | CFU/mL | CFU/mL | CFU/mL | CFU/mL |
| | 2 | >200K | >200K | >200K | >200K | 169K | 61K | 0 |
| | | CFU/mL | CFU/mL | CFU/mL | CFU/mL | CFU/mL | CFU/mL | CFU/mL |

Part 7—Immunoactive Activity of Transfer Factors Derived from a BLC

Although transfer factors do not have antimicrobial activity in and of themselves, this experiment demonstrates that the transfer factors produced in a BLC of the present invention can passively transfer immunity from one organism to another by stimulating cell mediated immunity. That is, transfer factor molecules are capable of transferring antigen specific targeting information. This experiment showed that the transfer factors produced in a BLC transfer antigen specific targeting information to defensins not previously exposed to the antigen, such that these defensins then exhibit antimicrobial activity against the antigen. The following method was used to test this proposition. The experiment was conducted under a sterile hood.

1. A sterile hood was prepared in the usual manner by 15 UV exposure and a washing with bleach.

2. Thereafter, 6 (10 cc) tubes of blood were drawn from a human volunteer (although bovine, or other mammalian, blood also could have been used). The whole blood in each of these tubes of blood was then divided into 15 cc Falcon sterile tubes (352096) and then spun at 250 RCF×15 min.

3. After spinning, the serum was carefully pipetted off, and the buffy coat was put into a separate tube.

4. The buffy coats were then combined into 2 tubes and the volume in each tube was then brought up to a volume of 10 cc with sterile normal saline (NS).

5. The tubes were then spun at 250 RCF×15 min.

6. Approximately 9.5 cc of liquid was carefully pipetted off, leaving the cells at the bottom.

7. The volume in each tube was then brought up to 10 cc with sterile NS.

8. Again, the tubes were spun at 250 RCF×15 min (third time).

9. Approximately 8.5 cc of liquid was carefully pipetted off, leaving the cells at the bottom.

10. Culture media was then prepared containing: 500 ul of 200 mML L-Glutamine; 5 ml of 10% Fetal Bovine Serum (Sigma F-2442); and RPMI 1640 (cellgro) to bring the total volume up to 50 cc. The final concentration of the L-glutamine was then 2 mML.

11. 15 cc of the above culture media was then placed into 3 separate culture flasks (i.e., Falcon Tissue Culture flask (353018)).

12. Into each flask, 1 cc of the lymphocyte cellular pellet from step 9 was added.

13. Thereafter, 10 ul of the sterile extract, reverse electrophoresed from band 12 (see above) from the uninfused sample (sample taken at the position of band 12 as there was no apparent band), traditionally infused sample or the gun inoculated sample (processed as described in Part 1) was drawn up and then placed into each flask. Therefore, the sterile extract was diluted by more than 1 to 1000 (since 10 ul placed into 10 cc of volume would lead to a dilution of 1 to 1000). Although, as shown in FIG. 1, transfer factors and defensins are both in band 12, our testing on extremely low concentrations of defensins as set forth in Table 6 below reveals that, at a 1 to 1000 dilution, defensins exhibit no antimicrobial activity. Consequently, whereas the dilution of the sterile extract in each flask was significantly beyond any level of antimicrobial activity exhibited by defensins, transfer factors were still active at this dilution as illustrated by the results provided in Table 7.

14. Each of the flasks was placed in a Napco CO₂ (5%) incubator at 37 C.

15. After 24 hrs, 2 cc of material was drawn up from each flask, was filtered through a Fisherbrand 0.2 micron filter (um) (09-719C), and then placed into a sterile bottle.

16. Thereafter, the defensin testing procedures set forth in Part 4 were used here to test for transfer factor induced defensin activity.

As suggested in step 13 above, when the material in band 12, which includes defensins, is diluted to 1000 to 1 (and even if diluted to a level of 200 to 1), defensins no longer exhibit any antimicrobial effect. This is illustrated in Table 6 below, where:

1=Sample 1 from un-infused cow

2=Sample 2 is from traditionally infused cow

3=Sample 3 is from gun inoculated cow

T=time in minutes

CFU/ml=colony forming units (in thousands) per 1 cc volume

1 ul Test Sample: 1000 ul SS=1:1001 (denoted in Table 6 as “1001”)

1 ul Test Sample: 500 ul SS=1:501 (denoted in Table 6 as “501”)

1 ul Test Sample: 200 ul SS=1:201 (denoted in Table 6 as “201”)

TABLE 6

| | | T₀ | T₁₅ | T₃₀ | T₄₅ | T₆₀ | T₇₅ | T₉₀ |
|---|---|---|---|---|---|---|---|---|
| 1001 | 1 | >200K | >200K | >200K | >200K | >200K | >200K | >200K |
| | | CFU/mL | CFU/mL | CFU/mL | CFU/mL | CFU/mL | CFU/mL | CFU/mL |
| | 2 | >200K | >200K | >200K | >200K | >200K | >200K | >200K |
| | | CFU/mL | CFU/mL | CFU/mL | CFU/mL | CFU/mL | CFU/mL | CFU/mL |
| | 3 | >200K | >200K | >200K | >200K | >200K | >200K | >200K |
| | | CFU/mL | CFU/mL | CFU/mL | CFU/mL | CFU/mL | CFU/mL | CFU/mL |
| 501 | 1 | >200K | >200K | >200K | >200K | >200K | >200K | >200K |
| | | CFU/mL | CFU/mL | CFU/mL | CFU/mL | CFU/mL | CFU/mL | CFU/mL |
| | 2 | >200K | >200K | >200K | >200K | >200K | >200K | >200K |
| | | CFU/mL | CFU/mL | CFU/mL | CFU/mL | CFU/mL | CFU/mL | CFU/mL |
| | 3 | >200K | >200K | >200K | >200K | >200K | >200K | >200K |
| | | CFU/mL | CFU/mL | CFU/mL | CFU/mL | CFU/mL | CFU/mL | CFU/mL |
| 201 | 1 | >200K | >200K | >200K | >200K | >200K | >200K | >200K |
| | | CFU/mL | CFU/mL | CFU/mL | CFU/mL | CFU/mL | CFU/mL | CFU/mL |
| | 2 | >200K | >200K | >200K | >200K | >200K | >200K | >200K |
| | | CFU/mL | CFU/mL | CFU/mL | CFU/mL | CFU/mL | CFU/mL | CFU/mL |
| | 3 | >200K | >200K | >200K | >200K | >200K | >200K | >200K |
| | | CFU/mL | CFU/mL | CFU/mL | CFU/mL | CFU/mL | CFU/mL | CFU/mL |

As shown in Table 7, the results of the transfer-factor induced antimicrobial activity tested for in this Part 7 of Example 3 were as follows:

The uninfused sample did not show any bactericidal activity.

The traditionally infused sample was bactericidal.

The gun-inoculated sample (i.e., a BLC) was highly bactericidal.

Note: the control samples which was un-infused material did not show any bactericidal activity and grew out positive>200K.

The following abbreviations are used in Table 7:

1=Sample 1—From traditionally infused cow.

2=Sample 2—From gun-inoculated cow (i.e., a BLC)

T=Minutes

10 ul Test Sample: 100 ul SS=1:11

5 ul Test Sample: 250 ul SS=1:51

5 ul Test Sample: 450 ul SS=1:91

TABLE 7

| | | T₀ | T₁₅ | T₃₀ | T₄₅ | T₆₀ | T₇₅ | T₉₀ |
|---|---|---|---|---|---|---|---|---|
| 91 | 1 | >200K | >200K | >200K | >200K | 184K | 101K | 28K |
| | | CFU/mL | CFU/mL | CFU/mL | CFU/mL | CFU/mL | CFU/mL | CFU/mL |
| | 2 | >200K | >200K | >200K | 79K | 41K | 12 | 0 |
| | | CFU/mL | CFU/mL | CFU/mL | CFU/mL | CFU/mL | CFU/mL | CFU/mL |
| 51 | 1 | >200K | >200K | >200K | 177K | 59K | 15K | 0 |
| | | CFU/mL | CFU/mL | CFU/mL | CFU/mL | CFU/mL | CFU/mL | CFU/mL |
| | 2 | >200K | 135K | 28K | 8K | 0 | 0 | 0 |
| | | CFU/mL | CFU/mL | CFU/mL | CFU/mL | CFU/mL | CFU/mL | CFU/mL |
| 11 | 1 | >200K | +/−200K | 47K | 0 | 0 | 0 | 0 |
| | | CFU/mL | CFU/mL | CFU/mL | CFU/mL | CFU/mL | CFU/mL | CFU/mL |
| | 2 | +/−200K | 22 | 0 | 0 | 0 | 0 | 0 |
| | | CFU/mL | CFU/mL | CFU/mL | CFU/mL | CFU/mL | CFU/mL | CFU/mL |

### Example 4

### Methods of Treatment Using a BLC

We envision that a BLC in various embodiments of the present invention that is prepared, for example, by the methods described in Examples 1 and 2, can be used as an antimicrobial and/or immunoactive composition to treat patients, as described in this example. It should be noted that the use of the present or future tense in this example (including each of its parts) is reflective of the fact that this example, including all of its parts, is prophetic. MPEP ¶ 608.01(p).

For example, we envision that, in embodiments of the present invention, a pharmaceutically effective amount of a BLC might be used in the treatment of acute infection (e.g., as described further in Part 1 below) or chronic infection to restore a patient's immune function (e.g., as described further in Part 2). Moreover, we envision that, in another embodiment of the present invention, a BLC could also impart an immunization effect (e.g., as described further in Part 3). However, in treating a patient, the antigen included in the antigen/cytokine mixture used to make a BLC should bear some resemblance, for example be essentially the same as or be exactly the same as the antigen with which the patient seeking treatment is afflicted (or against which the patient wishes to receive immunization) to be effective. The stronger the similarity between the antigen used to make a BLC and the antigen with which the patient seeking treatment is afflicted (or against which the patient wishes to receive immunization), the more effective a BLC is expected to be in treating the patient. In yet another embodiment of the present invention, a BLC or a blend of BLCs might be used to maintain, support and enhance the structure and function of the immune system in a healthy individual, as illustrated in Part 4 of this Example.

For example, an embodiment of a BLC might meet the following criteria. First, it can be filtered so that it contains peptides that are of 100 kD maximum molecular weight and less. Second, the BLC can demonstrate a “significant presence” of granulysins, transfer factors, and defensins, as evidenced by electrophoresis band density. In addition, lactoferrin also could be present. Third, as suggested above, the BLC can be found to be effective against the specific pathogen in question, such as Streptococcus pyogenes, in vitro, as described in Example 3.

If a BLC is found to be suitable, then an effective amount, for example, such as about 5 cc or less of liquid containing about 20% solids, 3 times daily, or about 1000 mg or less dried material daily, can be administered to a patient. If appropriate, the BLC can be formulated together with a pharmaceutically-acceptable, non-toxic carrier. The resulting product could be useable as a treatment modality for strep throat and could be administered by physician prescription after identification of the strep pathogen. As the material would utilize the subject's immune system and provide antimicrobial benefits, there would be no suppression of immune function. In fact, we envision that the subject's immune system would be enhanced and its performance elevated. Consequently, the material could be used for treatment and prevention.

### Part 1—Acute Infection

The following example describes a method of treating a patient with an acute infection using the BLC.

Subjective Data Obtained From Patient: An 8 year old boy presents with a 36 hour history of a fever up to 102.5 F, a left ear ache, nausea and a sore throat. By personal history, he has had several bouts of Strep throat. By social history, 4 other children in his class are also home sick with similar symptoms.

Objective Data Obtained From Patient (observation, tests, studies etc.): On physical examination, his temperature is found to be 101 F. H is left ear drum is found to be red and bulging. Examination of his throat shows a bilateral exudative tonsillitis with submandibular lymphadenopathy and his abdominal exam is benign.

Assessment of Patient's Status: An office “quick strep” test is positive for a Beta-hemolytic Strep and thus the clinician arrives at a diagnosis of “Strep Throat.”

Plan of Patient Care: A BLC according to an embodiment of the present invention prepared using Beta-hemolytic Strep as the antigen would be prescribed, in a liquid form, to be sprayed on the tonsils and in his ear four times per day. Appropriate supportive measures are also recommended.

Results: On follow-up examination of the patient three days later, the tonsillitis, lymphadenopathy and ear drum swelling is completely resolved, and the patient's appetite is back to normal. The patient is able to return to school.

### Part 2—Chronic Infection

In treating a patient with a chronic infection, we would expect that we could administer a BLC according to the present invention to impart a recovery by individuals from conditions that are caused by a dysfunctional or stressed immune system. For example, following a course of treatment, we would expect to see a broad effect upon the immune system as might be seen in blood tests that monitor immune system markers. (T&B cell subset, NK cell function test, TNF, Phagocytic Index).

Subjective Data Obtained From Patient: A 27 year old woman presents with an 8 year history of feeling chronically fatigued since having “mono” when she was in college. She also complains of insomnia, depression, dyslogia, dysmenorrhea and migratory myalgias.

Objective Data Obtained From Patient (observation, tests, studies etc.): Physical exam is non-contributory.

Assessment of Patient's Status: A summary of laboratory test abnormalities demonstrates the following.
1. Depressed levels of CD-19, CD-20, CD-21, CD-23 and HLA/DR-CD-20 lymphocytes.
2. EBV EA IgG and IgM antibodies are mildly elevated.
3. EBV EBNA antibodies are negative.
4. TSH is mildly elevated.
5. Mid afternoon and evening cortisol levels are elevated.
6. NK cell function is depressed.
   Thus a diagnosis of Chronic Fatigue Syndrome secondary to a chronic EBV infection is reached.

Plan of Patient Care: A BLC according to an embodiment of the present invention prepared using Epstein-Barr Virus as the antigen would be prescribed as (2) 100 mg capsules three times per day by mouth. Appropriate supportive measures and life style changes are also recommended.

Results:

6 Week Follow-Up Appointment:

The patient reports “feeling a little bit better.” She is falling asleep and staying asleep better. Her energy levels are still low but she feels more stable as are her moods. Her dyslogia and myalgias are unchanged.

A summary of laboratory test abnormalities demonstrate:
1. Depressed but improving levels of CD-19, CD-20, CD-21, CD-23 and HLA/DR-CD-20 lymphocytes.
2. EBV EA IgG and IgM antibody levels have almost doubled.
3. EBV EBNA IgM antibodies are now mildly elevated.
4. TSH is mildly elevated.
5. Mid afternoon and evening cortisol levels are closer to the normal range.
6. NK cell function are still depressed.

12 Week Follow-Up Appointment:

The patient reports “feeling a little bit better.” Her sleep cycle seems to have normalized. Her energy levels are still low but she feels more stable as are her moods. Her dyslogia is noticeably better but her myalgias are unchanged.

A summary of laboratory test abnormalities demonstrate:
1. Depressed but improving levels of CD-19, CD-20, CD-21, CD-23 lymphocytes. Her HLA/DR-CD-20 levels have increased to the bottom of the normal range.
2. EBV EA IgG and IgM antibody levels have almost tripled from her pre-treatment levels.
3. EBV EBNA IgM antibodies are now significantly elevated.
4. TSH is down to the high end of the normal range.
5. Mid afternoon and evening cortisol levels are down to the normal range.
6. NK cell function are still depressed but improving.

18 Week Follow-Up Appointment:

The patient reports “feeling much better.” Her sleep cycle seems to have normalized. Her energy levels are still low but she feels more stable as are her moods. Her dyslogia is noticeably better and her myalgias are improving.

A summary of laboratory test abnormalities demonstrates:
1. Her levels of CD-19, CD-20, CD-21, CD-23 lymphocytes are up to the bottom of the normal range. Her HLA/DR-CD-20 levels have increased to the middle of the normal range.
2. EBV EA IgG and IgM antibody levels have dropped to double from her pre-treatment levels.
3. EBV EBNA IgM antibodies are now significantly elevated and a EBV EBNA IgG antibody is now low level positive.
4. TSH is down to the high end of the normal range.
5. Mid afternoon and evening cortisol levels are still in the normal range.
6. NK cell function is up to the bottom of the normal range.

6 Month Follow-Up Appointment:

The patient reports “feeling much better.” Her sleep cycle is stable and normal. She describes consistently restful sleep. Her energy levels are still low but she feels more stable as are her moods. Her dyslogia is noticeably better and her myalgias are “much better”.

A summary of laboratory test abnormalities demonstrates:
1. Her levels of CD-19, CD-20, CD-21, CD-23 lymphocytes are up to the middle of the normal range. Her HLA/DR-CD-20 levels have increased to the high end of the normal range.
2. EBV EA IgG level remain elevated and the IgM antibody level has dropped to undetectable levels.
3. EBV EBNA IgM antibodies are still elevated but much lower and the EBV EBNA IgG antibody level are now double the previous level.
4. TSH is down to the middle of the normal range.
5. Mid afternoon and evening cortisol levels are still in the normal range.
6. NK cell function is up to the middle of the normal range.

8 Month Follow-Up Appointment:

The patient reports “feeling normal”. Her sleep cycle is still stable and normal. She described consistently restful sleep with normal dreaming. Her energy levels are back to normal as are her moods. Her dyslogia is noticeably better and her myalgias are “gone.”

A summary of laboratory test abnormalities demonstrate:
1. Her levels of CD-19, CD-20, CD-21, CD-23 lymphocytes are still up to the middle of the normal range. Her HLA/DR-CD-20 levels are still increased to the high end of the normal range.
2. EBV EA IgG level remain elevated but have dropped by 20% from the last test and the IgM antibody level is still down to undetectable levels.
3. EBV EBNA IgM antibodies are down to undetectable levels and the EBV EBNA IgG antibody level is now double the previous level of 2 months ago.
4. TSH is still down to the middle of the normal range.
5. Mid afternoon and evening cortisol levels are still in the normal range.
6. NK cell function is up to the high end of the normal range.

### Part 3—Immunization Effect

In administering a BLC of the present invention to a patient, we would expect that the BLC could impart a protective or immunization effect on the patient, such that the patient would be better able to combat colds, flu, food poisoning or other infections that normally would affect them as it does others in their environment. We envision that a BLC of the present invention product might be used in the immunization context as described below.

Subjective Data Obtained From Patient: A 47 year old man presents prior to going on a trip to India asking for a Hepatitis B vaccine. He is in “apparent good health” with no subjective complaints and no prior history of any form of Hepatitis.

Objective Data Obtained From Patient (observation, tests, studies etc.): Physical exam is non-contributory.

Assessment of Patient's Status: Hepatitis B vaccine should be administered.

Plan of Patient Care: The patient is offered and accepts a new, oral, non-toxic vaccine with no known side effects, namely, a BLC according to an embodiment of the present invention prepared using Hepatitis B Virus as the antigen). Accordingly, 100 mg of such a BLC would be prescribed to be taken once per day for 1 week.

Results:

6 Week Follow-Up:

He reports no adverse reactions. HBV antibody lab tests are done and found to be positive for the appropriate IgG antibodies.

### Part 4—Supporting the Structure and Function of the Immune System

In yet another embodiment of the present invention, a BLC or a blend of BLCs might be used to maintain, support and enhance the structure and function of the immune system in a healthy individual, as illustrated in this part.

Case 1

Nancy, a generally healthy thirty-four year old flight attendant misses several days of work each year due to the symptoms of colds and flu and seems to be vulnerable to such conditions even when her co-workers in the same environment are able to stay healthy. Due to her hectic schedule as a working mother, she feels stressed and tired much of the time. In her pursuit to improve her health, she begins reading health articles and talking to health-conscious friends. She learns that physical and emotional stress, if severe enough, can cause the immune system to be overworked, creating a situation that may cause a person to be more susceptible to colds and the flu. She goes to a health food store to shop for a supplement to support the immune system. The health food clerk recommends a dietary supplement made from a BLC according to an embodiment of the present invention, since the clerk has had feedback from a number of customers that this is an excellent dietary supplement for maintaining, supporting and enhancing the structure and function of the immune system. Nancy purchases this dietary supplement and takes the supplement daily along with her multivitamin. Over the course of the following year, she only misses one day of work and has half as many incidences of colds and flu, with the severity of those incidences being greatly reduced.

Case 2

Although generally healthy, Tommy nonetheless usually gets 5 or 6 colds a year, which is not uncommon for a second grader. Their neighbor, Mary, tells Tommy's mother, Harriet, about how her grandchildren seem to get fewer colds since they began taking a dietary supplement made from a BLC according to an embodiment of the present invention. Consequently, the next day, Harriet purchases such a dietary supplement for Tommy, which he ingests twice per day. During the next school year, while all of his classmates are getting their 5 or 6 colds, Tommy only gets two.

### Example 5

### Methicillin-Resistant Staphylococcus aureus (MRSA) Infective Dose and the Effective Dose of a Bio-Engineered Lacteal Complex (BLC) in a Murine, Septicemia Model

### Part 1—Experimental Overview

The purpose of the experiments described in this example was to determine the in vivo and in vitro activity of a Bioengineered Lacteal Complex (BLC) according to the present invention against a pathogen, specifically, Methicillin-Resistant Staphylococcus Aureus (MRSA). Detailed protocols utilized in the experiments are provided in Part 2 below.

Production of a BLC using MRSA as an antigen was prepared following the procedures outlined in Examples 1 and 2 above. After the gathering of milk from the cow was completed, the milk was processed as described in Example 2 to remove all material with a molecular weight greater than 100 kD. The 100 kD filtrate was then assayed according to the method outlined in Example 3 to determine if the applied process produced an antimicrobial effect relative to MRSA as contemplated according to the present invention in this application. From this in vitro testing, the BLC showed a very high kill rate relative to the MRSA strain selected and was determined to be viable for use with the in vivo mouse studies described below. The experimental design for these mouse studies required the creation of a number of Study Groups of mice, as further outlined in this Example.

At the outset, it was necessary to determine the “infective” dose for MRSA by testing the infecting activity of MRSA against several groups of mice (i.e., Study Groups A-E: 6 mice each, 3 male, 3 female), because mice have a high resistance to many pathogens. Typically, murine study models involve suppressing immune function to assure sickness of the test population without the initial testing needed for this study. However, according to our understanding, BLC employs immune function and immune competence. Consequently, we did not believe that immune suppression was appropriate for the mice in the study. The dose of 1×10⁹ CFU MRSA was determined to be the infective dose since, when the dose was administered to Study Group E (6 mice, 3 male, 3 female), all died within twelve (12) hours.

Also, initially it was necessary to determine the tolerated dose (i.e., treatment dose) of the BLC. Although several groups of mice were set aside in the experimental design to identify the tolerated dose of the BLC (e.g., Study Groups F-H: 6 mice each, 3 male, 3 female), testing using all of these study groups became unnecessary. The tolerated dose was efficiently determined by prior flow cytometry calculations and subsequent in vivo testing of the expected tolerated dose in Study Group F only. The Study Group F mice were observed for two additional weeks and no adverse effects were noted.

The tolerated dose identified from Study Group F was determined to be appropriate for the primary and secondary experimental studies forming part of the experimentation described in this Example involving, respectively, Study Groups J and I. Moreover, as a tertiary experimental study, the Study Group F mice were saved after the observation period and became Study Group K in order to attempt to demonstrate the ability of a BLC as an immunization agent by pre-treating mice with a second dose of the BLC prepared against MRSA ninety-six (96) hours (i.e., 4 days) before receiving an infective dose of MRSA. A more detailed discussion of the results and further discussion pertaining to the testing involving Study Groups I, J and K is provided, respectively, in Parts 3, 4 and 5 of this Example. However, a general overview of this testing and of the results is provided immediately below.

Study Group I was given the tolerated dose of the BLC established from the Study Group F analysis and then humanely sacrificed and their blood withdrawn, according to the Protocol provided in Part 2 of this Example, to further study of the pharmacokinetics of BLC. This pharmacokinetics work was done in vitro and allowed for a clearer understanding of the mechanisms of action of the BLC.

Study Group J included twenty-four (24) mice (12 male, 12 female). The twenty-four (24) mice were given the infective dose of MRSA (established from the Study Group A-E analysis) at 8 AM as well as the tolerated dose of the BLC (established from the Study Group F analysis) and another tolerated dose of the BLC four (4) hours later. The following briefly summarizes the results of the experiments.

1. By 12 noon of the same day, all of the mice had diarrhea and were at least mildly dehydrated. Four (4) of the male mice were more significantly impaired as evidenced by their lethargy and slow reaction to stimuli.

2. By 4 pm of the same day, twenty-two (22) of the mice were impaired, one (1) was moribund and one (1) was deceased.

3. Twenty-four (24) hours after the infective dose of MRSA, twenty (20) of the mice were normal, one (1) was impaired and two (2) more were deceased.

4. Forty-eight (48) hours after the infective dose, twenty of the mice were normal and one more was deceased.

5. The twenty (20) mice remaining were observed for another 5 days and then humanely sacrificed. There was no recurrence of symptoms and all twenty (20) mice appeared and behaved normally.

Study Group K was given a second tolerated dose (also referred to as a treatment dose) of the BLC ninety-six (96) hours (i.e., 4 days) before receiving an infective dose of MRSA. This followed their initial exposure to the tolerated dose as part of Study Group F. The following briefly summarizes the results of the experiments.

1. Four (4) hours after receiving the infective dose of MRSA, all Study Group K mice were impaired and one was moribund.

2. Eight (8) hours after receiving the infective dose of MRSA, two (2) mice were deceased and four (4) mice were impaired.

3. Twenty-four (24) hours after receiving the infective dose of MRSA, three (3) mice were impaired and one (1) more mouse was deceased.

4. Forty-eight (48) hours after receiving the infective dose of MRSA, the three remaining mice were normal and remained normal until the conclusion of the study one week from the start date.

In view of this testing, we reach the following general conclusions in this Example regarding the activity and efficacy of the BLC that was tested.

1. The BLC was highly effective in treating an in vivo MRSA infection. Twenty (20) of twenty-four (24) mice in Study Group J returned to normal within thirty-six (36) hours of receiving the infectious dose and remained normal.

2. The BLC provided an effective in vivo immunization response in Study Group K that caused a survival rate of 50% among the immunized mice that received the infective dose of MRSA.

3. The pharmacokinetic evaluation done with the blood of Study Group I demonstrated a clear and definite modulation curve. This provided an in vivo demonstration of a modulation effect created by the BLC, as discussed earlier in this specification.

4. The techniques and procedures according to the present invention are confirmed by in vivo study in this Example.

### Part 2—Experimental Protocol

As explained in Part 1 of this Example, mice in Study Groups A-E were used to determine the minimum infective dose of MRSA inoculum in an intra-peritoneal model. Mice in Study Groups F-H were intended to be used to determine the maximum tolerated dose of the BLC; however, only Study Group F ended up being used. Study Group I was used to demonstrate the blood-bioassay pharmacokinetics of the BLC. Study Group J was the group on which the statistical effectiveness of the BLC in the treatment of MRSA was tested. Finally, Study Group K was the group on which the immunization effects of the BLC were tested.

### (a) Identifying the Infective Dose of MRSA

The purpose of Study Groups A to E was to determine the infective dose of MRSA needed to induce severe incapacitation in 5 out of 6 or all of the mice in a study group.

Study Groups A-D

Each of Study Groups A to D was assembled so as to contain 6 BALB/c mice, 3 male and 3 female, non-immuno-compromised, as a peritoneal sepsis model.

Procedure in Each Study Group:
1. A betadine solution was applied to the abdomen of each mouse.
2. The following amounts of MRSA was injected into each mouse, starting with each mouse in Study Group A, and then in Study Group B, C and then D, with the results of the injection observed in each Study Group before proceeding to a higher amount of bacteria injected in a subsequent Study Group:
Study Group A: 1×10⁵ CFU MRSA, in log phase growth, was injected through a 27-gauge needle intra-peritoneally.
Study Group B: 1×10⁶ CFU MRSA, in log phase growth, was injected through a 27-gauge needle intra-peritoneally.
Study Group C: 1×10⁷ CFU MRSA, in log phase growth, was injected through a 27-gauge needle intra-peritoneally.
Study Group D: 1×10⁸ CFU MRSA, in log phase growth, was injected through a 27-gauge needle intra-peritoneally.

3. The animals were assessed at about 8 am, 12 noon, and 4 pm for signs of infection and their condition recorded.

4. The animals were observed for about seven days. No mouse became severely incapacitated in any of Study Groups A-D and all of the mice were humanely sacrificed.

Study Group E

Study Group E also was assembled to contain 6 BALB/c mice, 3 male and 3 female, non-immuno-compromised, as a peritoneal sepsis model. However, in this Study Group, since the MRSA concentrations injected in Study Groups A to D failed to kill any of the mice, the MRSA concentration used here was 1×10⁹ CFU MRSA, in log phase growth. As another modification, instead of suspending the bacteria in normal saline (NS), a 3% solution of Todd Hewitt Broth was used.

Procedure:

1. A betadine solution was applied to the abdomen of each mouse.

2. 1×10⁹ CFU MRSA, in log phase growth, was injected through a 27-gauge needle intra-peritoneally into each mouse.

3. The animals were assessed at about 8 am, 12 noon and 4 pm for signs of infection and their condition recorded.

4. The injection killed 100% of the six (6) mice that were injected within 12 hours. Thus, the bacterial formulation used on Study Group E was used in the testing conducted in Study Groups J and K.

### (b) Identifying the Tolerated/Treatment Dose of a BLC Against MRSA

The purpose of Study Groups F-H was to determine the maximum tolerated dose of the BLC. Although the experimental design was established to provide study groups to which incrementally increasing doses of the BLC was to be administered, only the protocol for Group F was carried out, as described below, since this was determined to be the tolerated/treatment dose of the BLC.

Study Group F

Materials: 6 BALB/c mice, 3 male and 3 female, non-immuno-compromised, in a peritoneal sepsis model.

Procedure:
1. A betadine solution was applied to the abdomen of each mouse.
2. 0.13 cc of BLC was injected through a 27-gauge needle intra-peritoneally in the left lower quadrant of each mouse.
3. The animals were assessed at about 8 am., 12 noon and 4 p.m. for signs of infection and their condition recorded.
4. The animals were observed for about two weeks and no apparent adverse effects were observed after two weeks of observation. No mouse became severely incapacitated.

### (c) Study Group Used for Pharmacokinetics Testing of the BLC

The mice in this study group were given the tolerated dose of the BLC (TD-BLC), only. The mice were then humanely sacrificed at hourly intervals and their blood collected such that pharmacokinetic testing could be conducted on the collected blood. Moreover, in vitro antimicrobial testing was also done by exposing the collected blood, at a 1:11 dilution, to various concentrations of MRSA.

Study Group I

Materials: 18 BALB/c mice, 9 male and 9 female, non-immuno-compromised, in a peritoneal sepsis model.

Procedure:

1. A betadine solution was applied to the abdomen of each mouse.

2. 100% of the tolerated dose of BLC (i.e., 0.13 cc) was given intra-peritoneally to each mouse (at time 0) (TO) and two (2) mice were anesthetized and sacrificed by exsanguinations and their blood collected immediately thereafter. The plasma was later separated from the whole blood and used in vitro, for example, in a 1:11 serial dilution, to help establish some of the pharmacokinetics and observed effects of the BLC by testing it against various concentrations of MRSA (i.e., 1×10⁸ CFU/ml, 1×10⁷ CFU/ml, 1×10⁶ CFU/ml and 1×10⁵ CFU/ml of the MRSA).

3. At every hour thereafter, starting at one (1) hour to eight (8) hours (i.e., at T1, T2, T3, T4, T5, T6, T7 and T8), two (2) mice were anesthetized and sacrificed by exsanguination and their blood collected. The plasma that was later separated from the whole blood (collected at each hour interval (i.e., at T1 to T8)) was used in vitro, for example in a serial dilution of 1:11, to help establish some of the pharmacokinetics and observed effects of the BLC by testing it in vitro against various concentrations of MRSA (i.e., 1×10⁸ CFU/ml, 1×10⁷ CFU/ml, 1×10⁶ CFU/ml and 1×10⁵ CFU/ml of the MRSA).

### (d) Study Group Used to Test the Therapeutic Efficacy of the BLC

The purpose of Study Group J was to determine the effectiveness of the tolerated/treatment dose of the BLC in a murine, septicemia model.

Study Group J

Materials: 12 BALB/c mice, 6 male and 6 female, non-immuno-compromised, in a peritoneal sepsis model.

Procedure:

1. A betadine solution was applied to the abdomen of each mice.

2. The infective dose of MRSA (i.e., 1×10⁹ CFU MRSA), in log phase growth, was injected into each mouse through a 27-gauge needle intra-peritoneally.

3. The tolerated/treatment dose of BLC (i.e., 0.13 cc) was administered to each mouse. A second dose of BLC was given 4 hours later to all the mice. Further doses of the tolerated dose of the BLC were given to mice that continued to be severely impaired.

4. The animals were assessed each day of the experiment, generally at about 8 am, 12 noon and 4 pm for signs of infection each day of the experiment and their condition recorded.

5. The animals were observed for about seven days and those that become severely incapacitated were humanely sacrificed.

6. The statistical efficacy of BLC was determined.

### (e) Study Group Used to Test the Immunization Effect of the BLC

The purpose of Study Group K was to determine the immunization effect of the BLC in a murine, septicemia model.

Study Grout K

Materials: 6 BALB/c mice, 3 male and 3 female, non-immuno-compromised, in a peritoneal sepsis model.

Procedure:

1. The six mice from Study Group F into which BLC was injected were taken and relabeled as mice in Study Group K. The day of this BLC injection became Day 1 of this experimental procedure.

2. On Day 30, a betadine solution was applied to the abdomen of each mouse, now in Study Group K, and each mouse was then re-dosed with the tolerated/treatment dose of BLC (i.e., 0.13 cc) and Todd Hewitt broth.

3. On Day 34, at 8 am, a betadine solution was applied to the abdomen of each mouse, and then each mouse was given a left lower quadrant intra-peritoneal injection of the infective dose of MRSA (i.e., 1×10⁹ CFU MRSA), in log phase growth, through a 27-gauge needle.

4. The animals were assessed thereafter each day of the experiment, generally at about 8 am, 12 noon and 4 pm for signs of infection each day of the experiment and their condition recorded.

5. The animals were observed for about seven days and those that become severely incapacitated were humanely sacrificed.

6. The statistical efficacy of BLC was determined.

### Part 3—Results and Further Testing and Analytical Procedures as to Study Group, I

This part describes the further testing that was conducted on the blood collected from the mice in Study Group I, as per the protocol set forth in Part 2(c) of Example 5. Two sets of experiments were conducted with this blood, as described further in Parts 3(a) and (b) set forth below, to better understand the pharmacokinetics and observed effects of the BLC used in this Example.

Materials/Instruments Required:
1. Sterile pipettes/pipettor tips
2. Sterile 1 cc culture tubes, should be polypropylene (from Sarstedt) with a gas permeable cap
3. Polystyrene tubes for the flow cytometry analysis
4. Biosafety hood
5. CO₂ incubator set at 37° C.
6. Phosphate Buffered Saline (PBS)
7. Flow Cytometer
8. “Green top” heparin tubes
9. CD stained antibodies
10. DPBS
11. FACSlyse

Further Analytical Procedure:
1. The blood from the Group I mice was collected as per the protocol provided in Part 2(c). In particular, the blood was collected from each mouse separately in a green top tube, in which 0.25 cc of phosphate buffered saline (i.e., PBS) was added first to put the heparin back into solution.
2. The whole blood from the 2 mice at each time point was then combined and was then tested for immune activation as per Part 3(a) below.
3. After the immune activation testing with the whole blood, it was centrifuged and the plasma tested for a bactericidal effect as described in Part 3(b) below.

### (a) Murine Immune Activation Experimental Design

The purpose of the first part of the experiment with the blood collected from the mice in Study Group I was to look for evidence of immune activation following an intra-peritoneal injection of the BLC in order to construct a pharmacokinetic or observed response curve. This immune activation was studied by measuring the level of activated leucocytes in the blood following BLC administration, pursuant to the following analytical protocol.

Further Analytical Procedure:
1. 10 ul of each of two antibodies that detect activated leucocytes (i.e., CD45PerCP, CD69PE) were mixed together and put into each of 9 small sterile polystyrene test tubes for a total of 20 ul of volume in each test tube (i.e., 9 test tubes of the experiment=90 ul of each antibody).
2. The whole blood (WB) collected from the Study Group I mice at each hour (i.e., from T0 to T8) was briefly vortexed to resuspend the cells.
3. 100 ul of the WB collected from each time interval (i.e., from T0 to T8) was placed, respectively, into each of 9 polystyrene tube and 20 ul of the antibody mix was added into each tube. Each of the 9 tubes was vortexed gently and then put in a dark place at room temperature for 0.15 min.
4. The whole tube rack containing the 9 test tubes was vortexed gently and 2 ml of FACSlyse was added into each tube. The whole tube rack was vortexed again and then put them into a dark place at room temperature for 15 minutes.
5. The whole tube rack was vortexed gently and centrifuged on “high” on a Sero-Fuge blood bank centrifuge for 3 minutes
6. The liquid from the tubes was “quickly dumped” out and the rim of the upside down tubes were blotted. The tubes were then turned right-side up and gently vortexed.
7. 2 ml of Phosphate Buffered Saline (PBS) was added.
8. The tubes were vortexed gently and centrifuged on “high” on a Sero-Fuge blood bank centrifuge for 3 minutes.
9. The liquid from the tubes was “quickly dumped” out and the rim of the upside down tubes were blotted. The tubes were then turned right-side up and gently vortexed.
10. 0.5 cc of Phosphate Buffered Saline (PBS) was added.
11. Cell viability was conducted using a standard iodine uptake test known to those having ordinary skill in the art to ensure accurate results and showed an index of 99% or better.
12. The cells were then analyzed in a flow cytometer to detect activated leucocytes.

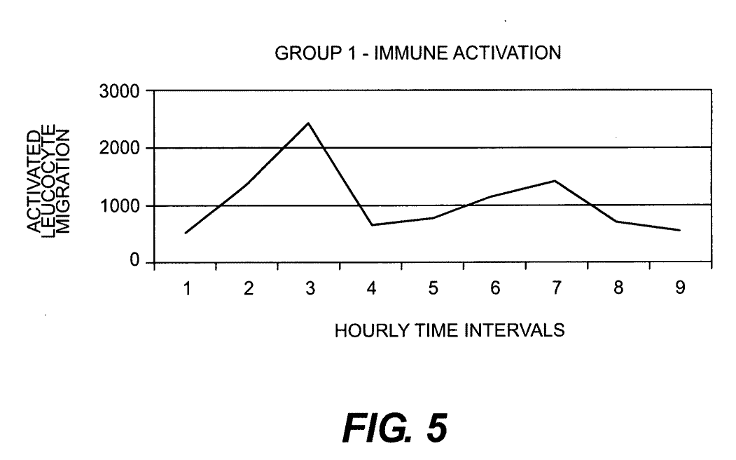

Results: The activation results demonstrated a significant production of various immunologically active cells, namely leucocytes, such as activated lymphocytes, large lymphocytes and natural killer (NK) cells. The table below reflects the number of such immunologically active cells found in the blood. These data points have been plotted in the graph provided in FIG. 5. We believe that these results, including the curve shown in FIG. 5, demonstrate that the BLC invokes an immune modulation response (the re-connection and activation of cytokine communication pathways to establish a new, higher, dynamic equilibrium of immune surveillance).

TABLE 8

| Time | Cell Count |
|---|---|
| 0 | 511 |
| 1 | 1351 |
| 2 | 2415 |
| 3 | 661 |
| 4 | 777 |
| 5 | 1126 |
| 6 | 1393 |
| 7 | 701 |
| 8 | 537 |

In particular, we believe that FIG. 5 shows the following:
1. Initially, hours 1 and 2 demonstrate immune stimulation; however, since we believe that the BLC provided targeting information but there was no real target (i.e., no MRSA was present), the immunological negative feedback pathways kicked in.
2. At hour 3, the feedback pathways have down regulated the immune response; however, as we believe is characteristic of an immune modulator (such as the BLC of the present invention), the trough is not as low as the initial level of immune response. That is, the BLC invokes an immune modulation response, namely, the re-connection and activation of cytokine communication pathways to establish a new, higher, dynamic equilibrium of immune surveillance.
3. At hours 4, 5 and 6, we observe a secondary immune stimulation response, but not as pronounced or rapid as the initial one. We would expect a pure immune stimulator to generally have but one peak of immune stimulation and then the molecules are enzymatically destroyed. (By immune stimulator, we mean any substance that can elicit an immune response, for example, such as INF-α, INF-β, IL-1, IL-2, IL-3, IL-4 etc.) However, a complex immune modulator, such as the BLC of the present invention, raises the overall level of surveillance through a cyclic pattern of stimulation and feedback to establish a new and higher dynamic equilibrium of immune function.
4. At hours 7 and 8, we again see feedback pathway activation that lowers immune function, but again not as low as the starting point.
5. Although such further experimentation was not conducted, it is hypothesized that, if more samples were taken, that one or more additional cycles would be seen with the level of immune function coming to rest at a level higher than it was at time 0.

### (b) Group I Murine Plasma In Vitro Antimicrobial Effects

This part of the Study Group I experimentation was designed to test for a bactericidal effect specific to the MRSA for which the BLC was made, using murine plasma following IP injection and collection at timed intervals.

Materials Required:
½ Normal Saline
TSA ll 5% Sheep Red Blood Cell (SRBC) Agar plate (BBL prepared media), 10 plates.
Processed murine blood to extract the plasma to be tested.
Active (in log phase) culture of a pathogenic strain of MRSA.
Small glass test tube for diluting the bacteria
1 ul culture loops
Vitek Colorimeter (needed to approximately standardize the concentration of bacteria to be tested). (The Colorimeter was calibrated by using the standard density controls as supplied by the manufacturer and by zeroing using ½ Normal Saline to align both ends of the instrument's scale.)

Procedure:
1. The bacteria to be tested was plated and grown on a standard TSA ll 5% sheep red blood cells (SRBC) Agar plate (BBL prepared media) and incubated in a CO2 Napco model 5400 incubator at 37 C and 5.0% CO2. The MRSA was repeatedly re-phenotyped through all phases of this research project (e.g., by way of an oxacillin plate and other methods known to those having ordinary skill in the art) to ensure strain consistency and lack of contamination. The original concentration of the bacteria was 1×10⁸ CFU/ml.
2. On the testing day, enough bacteria were added to a test tube of ½ normal saline (NS) (0.45% Baxter), with a sterile cotton swab, to decrease the solution to 95% transmissible on a Vitek Colorimeter (after being calibrated as above).
3. When the bacteria were added, the swab was twirled and the solution was allowed to settle before being measured. This became the stock solution (SS).
4. Murine whole blood (WB) was processed to extract the plasma and then was mixed with an appropriate volume (in a 1.5 cc capped micro centrifuge tube) of the SS to make a 1:11 dilutions (i.e., 10 ul Plasma: 100 ul SS=1:11).
5. The solutions were immediately plated (see below) (at time 0) and then put into the incubator until needed 15 min later. (The incubation time needed to be very short (i.e., 15 minute increments) in order to be able to reproducibly track any response).
6. After the time of incubation (and using a second incubated tube rack to take the needed tube out of the incubator so as to maintain as constant a temperature as is possible), a quantifying loop was utilized (1:1000, 1/1000 of a ml, 1 ul Nunc brand radiation sterilized, disposable) to take a sample (being careful to touch the loop to the inside of the tube to draw off any excess) of the mixture. This sample was then plated on ½ of a SRBC agar plate, the other 1 was plated with 1 ul of the SS or a second test product (prepared as above) and further incubated at 37 C.
7. After 18 hours, the dishes were removed from the incubator and “read”.
8. After reviewing these results, further testing was carried out holding the concentration of the plasma steady, serially diluting the concentration of the bacteria i.e., 1×10⁷, 1×10⁶ and 1×10⁵ CFU/ml, and repeating steps 4 to 7 above.

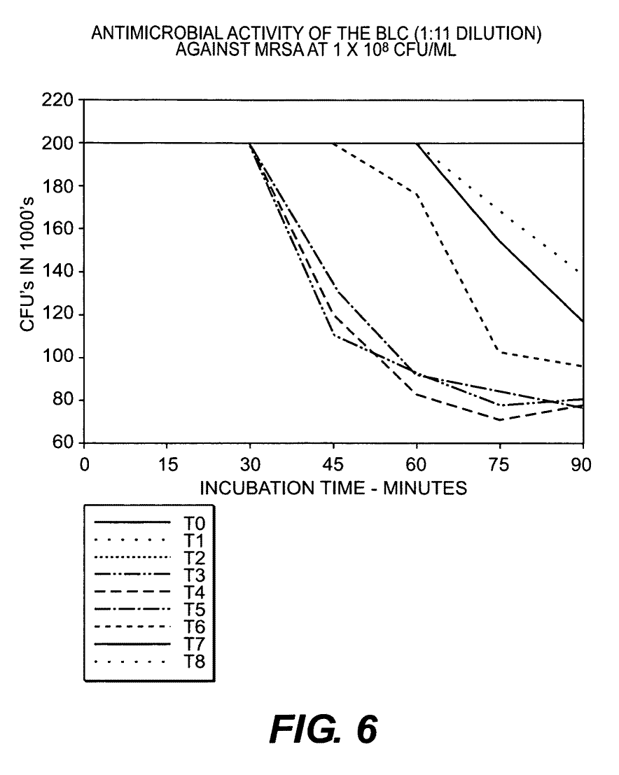

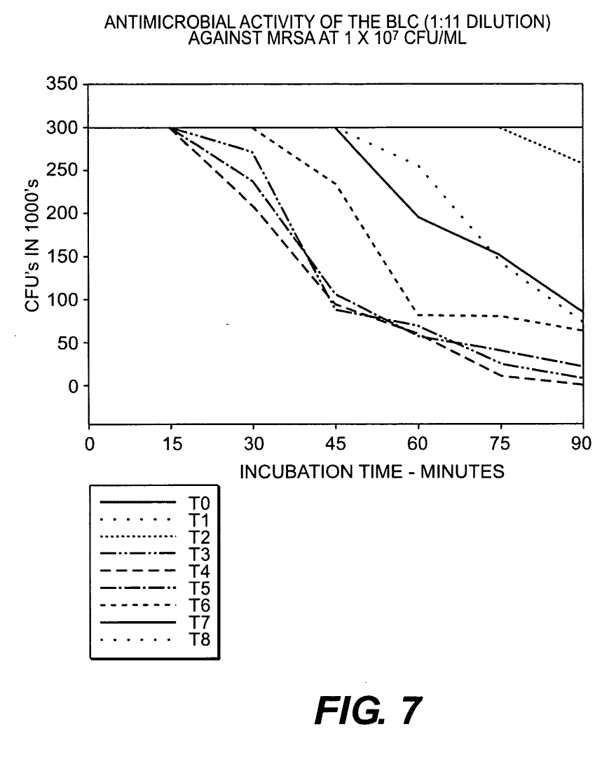

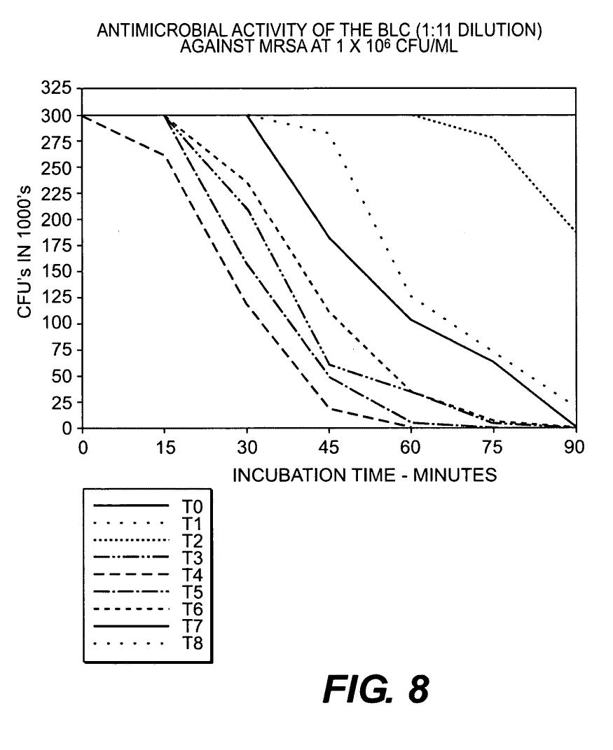

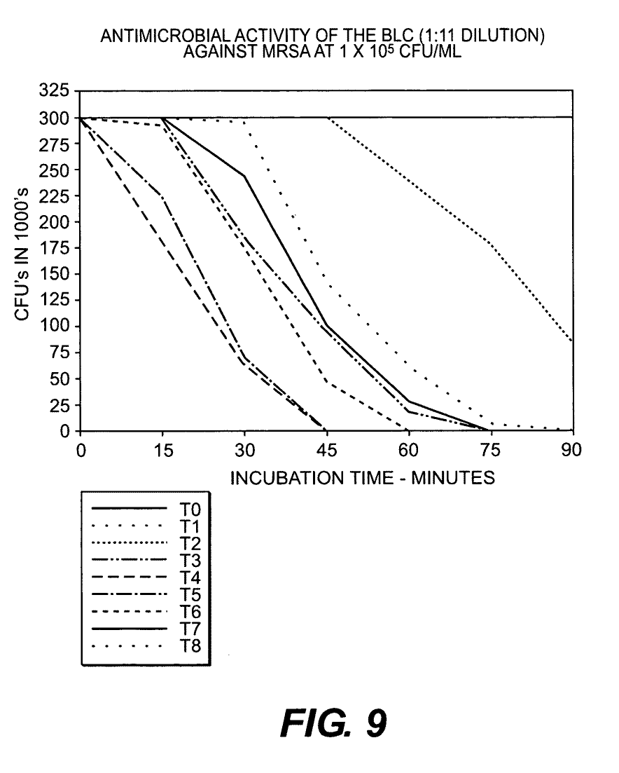

Results: See Tables 9 to 12 for numerical results of the experimentation conducted. FIGS. 6 to 9 provide a graphical depiction of the results of the experimentation. When reviewing Tables 9 to 12 and FIG. 6 to 9, it is important to recognize that T0 to T8 denote times at which mice were sacrificed by exsanguinations and their blood collected, as explained in the protocol provided in Part 2(c) of this example. Thereafter, the plasma, diluted to a 1:11 dilution, was incubated in vitro with varying bacterial concentrations of MRSA for increasing 15 minute increments (i.e., 15 min, 30 min, 45 min, 60 min, 75 min, 90 min) before undertaking the colony counts resulting in the data provided at Tables 9 to 12 and depicted in FIGS. 6 to 9.

TABLE 9
Antimicrobial Activity of the BLC (1:11 Dilution) Against
MRSA at 1 × 10⁸ CFU/ml

| Hour of | | | | | | | |
|---|---|---|---|---|---|---|---|
| Plasma | Number of CFU/ml at Each | | | | | | |
| Extraction | Noted Incubation Time (in 1000's) | | | | | | |
| Post the BLC | | 15 | | 45 | | 75 | |
| Administration | 0 min | min | 30 min | min | 60 min | min | 90 min |
| T0 | >200 | >200 | >200 | >200 | >200 | >200 | >200 |
| T1 | >200 | >200 | >200 | >200 | >200 | >200 | >200 |
| T2 | >200 | >200 | >200 | >200 | >200 | >200 | >200 |
| T3 | >200 | >200 | >200 | 111 | 93 | 78 | 81 |
| T4 | >200 | >200 | >200 | 120 | 83 | 71 | 78 |
| T5 | >200 | >200 | >200 | 134 | 92 | 84 | 77 |
| T6 | >200 | >200 | >200 | >200 | 176 | 103 | 96 |
| T7 | >200 | >200 | >200 | >200 | >200 | 154 | 117 |
| T8 | >200 | >200 | >200 | >200 | >200 | 168 | 139 |

TABLE 10
Antimicrobial Activity of the BLC (1:11 Dilution) Against
MRSA at 1 × 10⁷ CFU/ml

| Hour of | | | | | | | |
|---|---|---|---|---|---|---|---|
| Plasma | Number of CFU/ml at Each | | | | | | |
| Extraction | Noted Incubation Time (in 1000's) | | | | | | |
| Post BLC | | 15 | | 45 | | 75 | |
| Administration | 0 min | min | 30 min | min | 60 min | min | 90 min |
| T0 | >300 | >300 | >300 | >300 | >300 | >300 | >300 |
| T1 | >300 | >300 | >300 | >300 | >300 | >300 | >300 |
| T2 | >300 | >300 | >300 | >300 | >300 | >300 | 258 |
| T3 | >300 | >300 | 271 | 88 | 69 | 25 | 8 |
| T4 | >300 | >300 | 208 | 93 | 60 | 11 | 0 |
| T5 | >300 | >300 | 237 | 106 | 58 | 40 | 22 |
| T6 | >300 | >300 | >300 | 234 | 82 | 80 | 64 |
| T7 | >300 | >300 | >300 | >300 | 196 | 151 | 85 |
| T8 | >300 | >300 | >300 | >300 | 256 | 143 | 72 |

TABLE 11
Antimicrobial Activity of the BLC (1:11 Dilution) Against
MRSA at 1 × 10⁶ CFU/ml

| Hour of | | | | | | | |
|---|---|---|---|---|---|---|---|
| Plasma | Number of CFU/ml at Each | | | | | | |
| Extraction | Noted Incubation Time (in 1000's) | | | | | | |
| Post BLC | | 15 | | 45 | | 75 | |
| Administration | 0 min | min | 30 min | min | 60 min | min | 90 min |
| T0 | >300 | >300 | >300 | >300 | >300 | >300 | >300 |
| T1 | >300 | >300 | >300 | >300 | >300 | >300 | >300 |
| T2 | >300 | >300 | >300 | >300 | >300 | 277 | 187 |
| T3 | >300 | >300 | 210 | 61 | 34 | 4 | 0 |
| T4 | >300 | 261 | 118 | 18 | 0 | 0 | 0 |
| T5 | >300 | >300 | 157 | 49 | 4 | 0 | 0 |
| T6 | >300 | >300 | 236 | 111 | 35 | 6 | 0 |
| T7 | >300 | >300 | >300 | 182 | 103 | 63 | 0 |
| T8 | >300 | >300 | >300 | 282 | 126 | 73 | 19 |

TABLE 12
Antimicrobial Activity of the BLC (1:11 Dilution) Against
MRSA at 1 × 10⁵ CFU/ml

| Hour of | | | | | | | |
|---|---|---|---|---|---|---|---|
| Plasma | Number of CFU/ml at Each | | | | | | |
| Extraction | Noted Incubation Time (in 1000's) | | | | | | |
| Post BLC | | 15 | | 45 | | 75 | |
| Administration | 0 min | min | 30 min | min | 60 min | min | 90 min |
| T0 | >300 | >300 | >300 | >300 | >300 | >300 | >300 |
| T1 | >300 | >300 | >300 | >300 | >300 | >300 | >300 |
| T2 | >300 | >300 | >300 | >300 | 240 | 177 | 83 |
| T3 | >300 | >300 | 186 | 95 | 19 | 0 | 0 |
| T4 | >300 | 182 | 64 | 0 | 0 | 0 | 0 |
| T5 | >300 | 224 | 71 | 0 | 0 | 0 | 0 |
| T6 | >300 | 293 | 176 | 48 | 0 | 0 | 0 |
| T7 | >300 | >300 | 244 | 101 | 29 | 0 | 0 |
| T8 | >300 | >300 | 295 | 142 | 63 | 7 | 0 |

FIGS. 6 to 9 graphically depict the trend in the antimicrobial activity of the BLC of this Example. In summary, the in vitro results from the antimicrobial testing of the blood plasma of Study Group I mice obtained at various intervals following injection of the mouse with the BLC against MRSA reveals that the BLC had a strong antimicrobial or bactericidal effect. This marked antimicrobial effect was consistent even as the bacterial concentration was increased; the antimicrobial activity was unapparent in the first few hours post administration of the BLC (i.e., at about T0-T1 hours), peaking at about T3 to about T5 post administration and continuing its activity at T7 and T8 hours but less actively than before.

### Part 4—Results, Further Procedures and Analysis as to In Vivo Testing on Therapeutic Efficacy of the BLC in Treating MRSA-Infected Mice in Study Group J

Further to the experimental protocol set forth in Part 2(d) of this Example, at about 8 am on Day 1 of testing, the mice in Study Group J (i.e., 12 male and 12 female mice) were given a left lower quadrant intra-peritoneal injection of 1×10⁹ MRSA in Todd Hewitt broth. They were also given the tolerated dose of BLC (i.e., 0.13 cc) as a left lower quadrant intra-peritoneal injection. A second dose of BLC was given 4 hours later to all the mice (based upon the flow cytometry curve obtained from testing on Study Group I, described in more detail in Part 3 of this Example, and depicted in FIG. 5. Further doses of the tolerated dose of BLC were given to mice that continued to be severely impaired, as noted in the “Comments” column of Table 13 below. Repeat dosing of the BLC was not automatically given because of the concern of trauma from the injection itself. Our observations during the course of this experiment are provided in Table 13 below.

Table 13 shows that 83% (20 out of 24) of the mice survived a left lower quadrant intra-peritoneal injection of 1×10⁹ MRSA in Todd Hewitt broth. The mice given this same dose without prior dosing of BLC and without concurrent dosing of BLC had a 100% mortality.

TABLE 13

| | Mouse | Mouse | Mouse | Mouse | |
|---|---|---|---|---|---|
| Observation | Status: | Status: | Status: | Status: | |
| Time | Normal | Impaired | Moribund | Deceased | Comments |
| Day 1 | 12 Male | — | — | — | — |
| 8 am | 12 Female | | | | |
| Day 1 | — | 12 Male | — | — | By noon all of the mice had |
| 12 noon | | 12 Female | | | diarrhea and were at least |
| | | | | | mildly dehydrated. Four of the |
| | | | | | male mice were more |
| | | | | | significantly impaired as |
| | | | | | evidenced by their lethargy and |
| | | | | | slow reaction to stimuli. |
| Day 1 | — | 10 Male | 1 Male | 1 Male | All of the mice had diarrhea |
| 4 pm | | 12 Female | | | and were at least mildly |
| | | | | | dehydrated. Two of the male |
| | | | | | mice and one of the female |
| | | | | | mice were more significantly |
| | | | | | impaired as evidenced by their |
| | | | | | lethargy and slow reaction to |
| | | | | | stimuli and were re-injected |
| | | | | | with BLC. |
| Day 2 | 8 Male | 1 Male | — | 2 Male | The one impaired mouse was |
| 8 am | 12 Female | | | | re-injected with BLC. |
| Day 2 | 7 Male | 2 Male | — | — | The two impaired mice were |
| 12 noon | 12 Female | | | | re-injected with BLC. |
| Day 2 | 7 Male | 2 Male | — | — | The two impaired mice were |
| 4 pm | 12 Female | | | | re-injected with BLC. |
| Day 3 | 8 Male | — | — | 1 Male | — |
| 8 am | 12 Female | | | | |
| Day 3 | 8 Male | — | — | — | — |
| 12 noon | 12 Female | | | | |
| Day 3 | 8 Male | — | — | — | — |
| 4 pm | 12 Female | | | | |
| Day 4 | 8 Male | — | — | — | — |
| 8 am | 12 Female | | | | |
| Day 4 | 8 Male | — | — | — | — |
| 12 noon | 12 Female | | | | |
| Day 4 | 8 Male | — | — | — | — |
| 4 pm | 12 Female | | | | |
| Day 5 | 8 Male | — | — | — | — |
| 8 am | 12 Female | | | | |
| Day 6 | 8 Male | — | — | — | — |
| 8 am | 12 Female | | | | |
| Day 7 | 8 Male | — | — | — | — |
| 7 am | 12 Female | | | | |
| Day 7 | 8 Male | — | — | — | — |
| 12 noon | 12 Female | | | | |
| Day 7 | 8 Male | — | — | — | — |
| 4 pm | 12 Female | | | | |
| Day 8 | 8 Male | — | — | — | After the 7 am observation all |
| 7 am | 12 Female | | | | of the mice were humanely |
| | | | | | euthanized. |

### Part 5—Results, Further Procedures and Analysis as to In Vivo Testing on Immunization Effect of the BLC in on Mice in Study Group K

Further to the experimental protocol set forth in Part 2(e) of this Example, Group F was injected with the BLC (e.g., referred to Day 1 in this part of the experiments conducted in this Example) and tolerated it without any apparent change to their health or behavior. They were kept and re-labeled as Group K to test for an immunization effect that was hypothesized based upon in-vitro flow cytometry data. They were re-dosed with a combination of BLC (full dose) and Todd Hewitt broth (to test for toxicity) on Day 30 and again had no apparent adverse effects to their health or behavior. On Day 34, at 8 am, 3 male and 3 female mice were given a left lower quadrant intra-peritoneal injection of 1×10⁹ MRSA in Todd Hewitt broth only, and no more BLC was given. Our observations during the course of this experiment are provided in Table 14 below.

Table 14 shows that 50% of the mice had survived a left lower quadrant intra-peritoneal injection of 1×10⁹ MRSA in Todd Hewitt broth. The mice given this same dose without prior dosing of BLC and without concurrent dosing of BLC had a 100% mortality.

TABLE 14

| | Mouse | Mouse | Mouse | Mouse | |
|---|---|---|---|---|---|
| Observation | Status: | Status: | Status: | Status: | |
| Time | Normal | Impaired | Moribund | Deceased | Comments |
| Day 34 | 3 Male | — | — | — | — |
| 8 am | 3 Female | | | | |
| Day 34 | — | 3 Male | 1 Female | — | By noon all of the mice had |
| 12 noon | | 2 Female | | | diarrhea and were at least |
| | | | | | mildly dehydrated. |
| Day 34 | — | 3 Male | 2 Female | — | The two moribund mice |
| 4 pm | | 1 Female | | | were humanely euthanized |
| | | | | | at 4 pm. |
| Day 35 | — | 2 Male | — | 1 Male | — |
| 8 am | | 1 Female | | | |
| Day 35 | 1 Male | 1 Male | — | — | — |
| 12 noon | 1 Female | | | | |
| Day 35 | 1 Male | 1 Male | — | — | — |
| 4 pm | 2 Female | | | | |
| Day 36 | 2 Male | — | — | — | — |
| 8 am | 1 Female | | | | |
| Day 36 | 2 Male | — | — | — | — |
| 12 noon | 1 Female | | | | |
| Day 36 | 2 Male | — | — | — | — |
| 4 pm | 1 Female | | | | |
| Day 37 | 2 Male | — | — | — | — |
| 8 am | 1 Female | | | | |
| Day 37 | 2 Male | — | — | — | — |
| 12 noon | 1 Female | | | | |
| Day 37 | 2 Male | — | — | — | — |
| 4 pm | 1 Female | | | | |
| Day 38 | 2 Male | — | — | — | — |
| 8 am | 1 Female | | | | |
| Day 39 | 2 Male | — | — | — | — |
| 8 am | 1 Female | | | | |
| Day 40 | 2 Male | — | — | — | — |
| 7 am | 1 Female | | | | |
| Day 40 | 2 Male | — | — | — | — |
| 12 noon | 1 Female | | | | |
| Day 40 | 2 Male | — | — | — | — |
| 4 pm | 1 Female | | | | |
| Day 41 | 2 Male | — | — | — | After the 7 am observation |
| 7 am | 1 Female | | | | all of the mice were |
| | | | | | humanely euthanized. |

While particular embodiments of the present invention have been illustrated and described, it will be apparent to those skilled in the art that various changes and modifications may be made without departing from the spirit and scope of the invention. Furthermore, it is intended that the claims will cover all such modifications that are within the scope of the invention.

1. A composition that is capable of providing an antimicrobial benefit and/or reestablishing cytokine pathways when administered to a living organism in a pharmaceutically effective dose, wherein the composition comprises:
a pharmaceutically effective combination comprising: (i) an amount of granulysins; (ii) an amount of transfer factors; (iii) an amount of defensins; and (iv) an amount of mini-cytokines, wherein the granulysins, transfer factors and defensins are specific to a selected pathogen; and a pharmaceutically acceptable carrier.

2-40. (canceled)
